(19) United States
(12) Patent Application Publication
(10) Pub. No.: US 2024/0062902 A1
(43) Pub. Date: Feb. 22, 2024

(54) CHRONIC PULMONARY DISEASE PREDICTION FROM AUDIO INPUT BASED ON SHORT-WINDED BREATH DETERMINATION USING ARTIFICIAL INTELLIGENCE

(71) Applicant: SONY GROUP CORPORATION, TOKYO (JP)

(72) Inventors: SACHIN KUMAR AGRAWAL, BENGALURU (IN); AYUSH KUMAR GHADIYA, BENGALURU (IN)

(21) Appl. No.: 18/364,078

(22) Filed: Aug. 2, 2023

Related U.S. Application Data
(60) Provisional application No. 63/371,569, filed on Aug. 16, 2022.

(51) Int. Cl. G16H 50/20; G10L 25/66; G10L 25/93

(52) U.S. Cl. CPC G16H 50/20; G10L 25/66; G10L 25/93

(57) ABSTRACT

An electronic device and method for chronic pulmonary disease prediction from audio input based on short-winded breath determination using artificial intelligence is disclosed. The electronic device receives an audio input associated with a user. The electronic device applies an Artificial Intelligence (AI) model to detect a short-winded breath duration that corresponds to a time duration between an end of a first spoken word and a start of a second spoken word succeeding the first spoken word. The electronic device detects a speaking pattern. The electronic device applies a Recurrent neural network (RNN) model to reconstruct a set of short-winded breath audio samples. The electronic device generates an audio sample dataset and a set of audio features. The electronic device applies a modular neural network model on the generated audio sample dataset and on the generated set of audio features to determine a set of chronic obstructive pulmonary disease (COPD) metrics.

### CROSS-REFERENCE TO RELATED APPLICATIONS/INCORPORATION BY REFERENCE

This Application also makes reference to U.S. Provisional Application Ser. No. 63/371,569, which was filed on Aug. 16, 2022. The above stated Patent Applications are hereby incorporated herein by reference in their entirety

### FIELD

Various embodiments of the disclosure relate to chronic pulmonary disease prediction. More specifically, various embodiments of the disclosure relate to an electronic device and method for chronic pulmonary disease prediction from audio input based on short-winded breath determination using artificial intelligence.

### BACKGROUND

Industrial growth and increased vehicular traffic have led to an increase in respiratory diseases, such as, asthma, chronic obstructive pulmonary disease (COPD), and the like. Such diseases may be mainly caused due to air pollution, smoking, agricultural pesticides, and industrial chemicals. The symptoms of COPD may be breathlessness, frequent coughing with or without sputum, wheezing and tightness in the chest. Bronchitis and Emphysema have frequently been used as markers for diagnosis of COPD. In cases, COPD may also be caused due to long-term asthma. COPD and asthma may have detrimental effects in a patient's quality of life and may hinder the patient from performance of daily life activities. Clinically, diseases such as, asthma and COPD, may be suspected based on a clinical history and medical examination of the patient, but a confirmation of the diagnosis such diseases may be done by spirometry. Spirometry is a commonly used test to measure an amount of air that enters and leaves the lungs, before and after a use of an inhaled bronchodilator. However, spirometry does not provide etiological diagnosis, fails to detect obstructive-restrictive defect, depends on the patient's effort, and requires skilled operators. Moreover, spirometry may be costly and cause annoyance for the patients as diagnosis with spirometry may require a multiple number of breathing maneuvers.

Limitations and disadvantages of conventional and traditional approaches will become apparent to one of skill in the art, through comparison of described systems with some aspects of the present disclosure, as set forth in the remainder of the present application and with reference to the drawings.

### SUMMARY

An electronic device and method for chronic pulmonary disease prediction from audio input based on short-winded breath determination using artificial intelligence, and/or described in connection with, at least one of the figures, as set forth more completely in the claims.

These and other features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

FIG. 1 is a block diagram that illustrates an exemplary network environment for chronic pulmonary disease prediction from audio input based on short-winded breath determination using artificial intelligence, in accordance with an embodiment of the disclosure.

FIG. 2 is a block diagram that illustrates an exemplary electronic device of FIG. 1, in accordance with an embodiment of the disclosure.

FIGS. 3A and 3B are diagrams that collectively illustrate an exemplary processing pipeline for chronic pulmonary disease prediction from audio input based on short-winded breath determination using artificial intelligence, in accordance with an embodiment of the disclosure.

FIGS. 4A and 4B are diagrams that together illustrate an exemplary scenario for chronic pulmonary disease prediction from audio input based on short-winded breath determination using artificial intelligence, in accordance with an embodiment of the disclosure.

FIG. 5 is a flowchart that illustrates operations of an exemplary method for denoising audio input, in accordance with an embodiment of the disclosure.

FIG. 6A is a diagram that illustrates an exemplary scenario for detection of short-winded breath duration, in accordance with an embodiment of the disclosure.

FIG. 6B is a diagram that illustrates an exemplary scenario for detection of short-winded breath duration for a selected user, in accordance with an embodiment of the disclosure.

FIG. 6C is a diagram that illustrates an exemplary scenario for detection of short-winded breath duration for a plurality of users, in accordance with an embodiment of the disclosure.

FIG. 7 is a diagram that illustrates an exemplary processing pipeline for reconstruction of a set of short-winded breath audio samples based on application of Recurrent Neural Network (RNN) model, in accordance with an embodiment of the disclosure.

FIG. 8 is a diagram that illustrates an exemplary scenario for application of generative cyclic autoencoder modular neural network, in accordance with an embodiment of the disclosure.

FIG. 9 is a diagram that illustrates an exemplary processing pipeline for statistical data generation using statistical generative adversarial network model, in accordance with an embodiment of the disclosure.

FIG. 10 is a diagram that illustrates an exemplary processing pipeline for statistical data generation using statistical generative adversarial network model, in accordance with an embodiment of the disclosure.

FIG. 11A is a diagram that illustrates an exemplary scenario of cyclic deep contractive autoencoder model, in accordance with an embodiment of the disclosure.

FIG. 11B is a diagram that illustrates an exemplary scenario of cyclic deep contractive autoencoder model with minimum losses, in accordance with an embodiment of the disclosure.

FIG. 12 is a flowchart that illustrates operations of an exemplary method for dimensionality reduction using cyclic contractive autoencoder model, in accordance with an embodiment of the disclosure.

FIG. 13 is a diagram that illustrates an exemplary scenario for continuous monitoring of a user using artificial intelligence, in accordance with an embodiment of the disclosure.

FIG. 14 is a diagram that illustrates an exemplary processing pipeline for continuous monitoring of a user using artificial intelligence, in accordance with an embodiment of the disclosure.

FIG. 15 is a diagram that illustrates an exemplary processing pipeline for encryption and decryption of the reconstructed set of short-winded breath audio samples, in accordance with an embodiment of the disclosure.

FIG. 16 is a flowchart that illustrates operations of an exemplary method for chronic pulmonary disease prediction from audio input based on short-winded breath determination using artificial intelligence, in accordance with an embodiment of the disclosure.

### DETAILED DESCRIPTION

The following described implementation may be found in an electronic device and a method for chronic pulmonary disease prediction from an audio input based on a short-winded breath determination using artificial intelligence. Exemplary aspects of the disclosure may provide an electronic device (for example, a server, a desktop, a laptop, or a personal computer) that may receive an audio input associated with a user. The electronic device may apply an Artificial Intelligence (AI) model on the received audio input. The electronic device may detect a short-winded breath duration associated with the received audio input, based on the application of the AI model on the received audio input. The short-winded breath duration may correspond to a time duration between an end of a first spoken word and a start of a second spoken word succeeding the first spoken word in the received audio input. The electronic device may detect a speaking pattern associated with the received audio input, based on the application of the AI model on the received audio input and a geolocation of the user. The electronic device may apply a recurrent neural network (RNN) model (for example, a variational RNN, an Attention Peephole RNN) on audio samples associated with the received audio input, based on the detected short-winded breath duration and the detected speaking pattern. The electronic device may reconstruct a set of short-winded breath audio samples based on the application of the RNN model on the audio samples associated with the received audio input. The electronic device may generate an audio sample dataset and a set of audio features associated with the generated audio sample dataset, based on a statistical analysis of the reconstructed set of short-winded breath audio samples. The electronic device may apply a modular neural network model on the generated audio sample dataset and on the generated set of audio features. The electronic device may determine a set of chronic obstructive pulmonary disease (COPD) metrics associated with the user, based on the application of the modular neural network model on the generated audio sample dataset and the generated set of audio features.

Typically, a diagnosis of the COPD is done by a spirometry. It may be appreciated that the spirometry may diagnose the COPD based on a measurement of an amount of air that enters and leaves the lungs, before and after a use of an inhaled bronchodilator. However, spirometry does not provide etiological diagnosis, fails to detect obstructive-restrictive defect, provides diagnosis based on a patient's effort, and requires skilled operators. Moreover, spirometry may be costly and may cause annoyance to the patients as diagnosis with spirometry may require a multiple number of breathing maneuvers.

In order to non-invasively diagnose the COPD, the present disclosure introduces a method of chronic pulmonary disease prediction from audio input based on short-winded breath determination using artificial intelligence. The electronic device of the present disclosure detects a COPD condition based on respiratory sounds. As, sound produced by a patient's internal organs may be different in case of a heart attack, asthma, COPD, and the like, automated detection of such sounds may be used to predict if the patient is susceptible to COPD. Further, a prediction of COPD based on the short-winded breath duration may be a time-saving and cost-effective method for both the patient such as, a user, and a healthcare professional such as, a doctor. Moreover, the disclosed electronic device may detect different speaking patterns and may not get biased based on the different audio samples from users of different geographical regions. Based on the different geographical regions, the AI model of the present disclosure may treat each sample as normal or abnormal and may classify them accordingly. Furthermore, the disclosed electronic device may continuously monitor the patient, such as, the user, and may record a set of audio inputs associated with the patient when a COPD condition is detected. The disclosed method may be performed by smart devices (such as, a portable Internet of Things (IoT)-enabled breath sensor, a smart phone, and the like) to record the set of audio inputs associated with the patient routinely and predict a possibility of COPD.

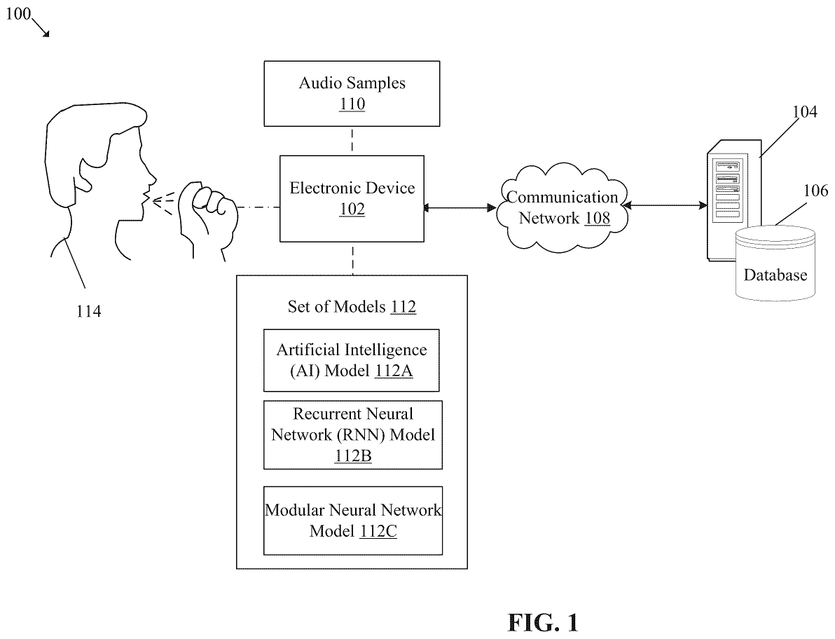

FIG. 1 is a block diagram that illustrates an exemplary network environment for chronic pulmonary disease prediction from audio input based on short-winded breath determination using artificial intelligence, in accordance with an embodiment of the disclosure. With reference to FIG. 1, there is shown a network environment 100. The network environment 100 may include an electronic device 102, a server 104, a database 106, and a communication network 108. In FIG. 1, there is further shown audio samples 110. The electronic device 102 may be associated with a set of models 112. The set of models 112 may include an Artificial Intelligence (AI) model 112A, a Recurrent neural network (RNN) model 112B, and a modular neural network model 112C. The electronic device 102 and the server 104 may be communicatively coupled to each another, via the communication network 108. In FIG. 1, there is further shown a user 114 who may be associated with and/or may operate the electronic device 102.

The electronic device 102 may include suitable logic, circuitry, interfaces, and/or code that may be configured to receive an audio input associated with a user, such as, the user 114. The electronic device 102 may apply the Artificial Intelligence (AI) model 112A on the received audio input. The electronic device 102 may detect a short-winded breath duration associated with the received audio input, based on the application of the AI model 112A on the received audio input. The short-winded breath duration may correspond to a time duration between an end of a first spoken word and a start of a second spoken word succeeding the first spoken word in the received audio input. The electronic device 102 may detect a speaking pattern associated with the received audio input, based on the application of the AI model 112A on the received audio input and a geolocation of the user 114. The electronic device 102 may apply the Recurrent neural network (RNN) model 112B on audio samples associated with the received audio input, based on the detected short-winded breath duration and the detected speaking pattern. The electronic device 102 may reconstruct a set of short-winded breath audio samples based on the application of the RNN model 112B on the audio samples associated with the received audio input. Examples of the electronic device 102 may include, but are not limited to, a computing device, a smartphone, a cellular phone, a mobile phone, a gaming device, a mainframe machine, a server, a computer workstation, and/or a consumer electronic (CE) device.

The server 104 may include suitable logic, circuitry, and interfaces, and/or code that may be configured to generate an audio sample dataset and a set of audio features associated with the generated audio sample dataset, based on a statistical analysis of the reconstructed set of short-winded breath audio samples. The server 104 may apply the modular neural network model 112C on the generated audio sample dataset and on the generated set of audio features. The server 104 may determine a set of chronic obstructive pulmonary disease (COPD) metrics associated with the user 114, based on the application of the modular neural network model 112C on the generated audio sample dataset and the generated set of audio features.

In one or more embodiments, the server 104 may store the set of models 112, the generated audio sample dataset, and/or the set of audio features associated with the generated audio sample dataset. Further, the server 104 may execute at least one operation associated with the electronic device 102. The server 104 may be implemented as a cloud server and may execute operations through web applications, cloud applications, HTTP requests, repository operations, file transfer, and the like. Other example implementations of the server 104 may include, but are not limited to, a database server, a file server, a web server, a media server, an application server, a mainframe server, or a cloud computing server.

In at least one embodiment, the server 104 may be implemented as a plurality of distributed cloud-based resources by use of several technologies that are well known to those ordinarily skilled in the art. A person with ordinary skill in the art will understand that the scope of the disclosure may not be limited to the implementation of the server 104 and the electronic device 102, as two separate entities. In certain embodiments, the functionalities of the server 104 can be incorporated in its entirety or at least partially in the electronic device 102 without a departure from the scope of the disclosure. In certain embodiments, the server 104 may host the database 106. Alternatively, the server 104 may be separate from the database 106 and may be communicatively coupled to the database 106.

The database 106 may include suitable logic, interfaces, and/or code that may be configured to store the set of chronic obstructive pulmonary disease (COPD) metrics. The database 106 may be stored or cached on a device, such as a server (e.g., the server 104) or the electronic device 102. The device storing the database 106 may be configured to receive (e.g., from the electronic device 102 and/or the server 104) a query for the set of chronic obstructive pulmonary disease (COPD) metrics. In response, the device that stores the database 106 may retrieve and provide the set of chronic obstructive pulmonary disease (COPD) metrics to the electronic device 102 and/or the server 104.

In some embodiments, the database 106 may be hosted on a plurality of servers stored at same or different locations. The operations of the database 106 may be executed using hardware, including a processor, a microprocessor (e.g., to perform or control performance of one or more operations), a field-programmable gate array (FPGA), or an application-specific integrated circuit (ASIC). In some other instances, the database 106 may be implemented using software.

The communication network 108 may include a communication medium through which the electronic device 102 and the server 104 may communicate with each another. The communication network 108 may be one of a wired connection or a wireless connection. Examples of the communication network 108 may include, but are not limited to, the Internet, a cloud network, Cellular or Wireless Mobile Network (such as Long-Term Evolution and 5th Generation (5G) New Radio (NR)), a satellite network (such as, a network of a set of Low Earth Orbit satellites), a Wireless Fidelity (Wi-Fi) network, a Personal Area Network (PAN), a Local Area Network (LAN), or a Metropolitan Area Network (MAN). Various devices in the network environment 100 may be configured to connect to the communication network 108 in accordance with various wired and wireless communication protocols. Examples of such wired and wireless communication protocols may include, but are not limited to, at least one of a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity (Li-Fi), 802.16, IEEE 802.11s, IEEE 802.11g, multi-hop communication, wireless access point (AP), device to device communication, cellular communication protocols, and Bluetooth (BT) communication protocols.

The AI model 112A may include suitable logic, interfaces, and/or code that may be configured to detect the speaking pattern associated with the received audio input based on the received audio input and the geolocation of the user 114. In an embodiment, the AI model 112A may correspond to a Self-Correcting Artificial Neural Network (SCANN) model. The SCANN model may include a self-correcting layer. The SCANN model may provide an output irrespective of a characteristics of an input to the SCANN model.

The RNN model 112B may include suitable logic, interfaces, and/or code that may be configured to reconstruct the set of short-winded breath audio samples based on the audio samples 110 associated with the received audio input. The RNN model 112B may be a recurrent neural network model that may be capable of analysis of sequential or time series data as the RNN model 112B may be configured to learn long-term dependencies in the sequential or time series data.

The modular neural network model 112C may include suitable logic, interfaces, and/or code that may be configured to determine the set of chronic obstructive pulmonary disease (COPD) metrics associated with the user 114, based on the generated audio sample dataset and the generated set of audio features. In an embodiment, the modular neural network model 112C may further configured to classify a health condition associated with the user as one of a COPD condition or a non-COPD condition. The COPD condition may be used to determine that the user 114, associated with the received audio input, may be prone to or currently suffer from COPD. The non-COPD condition may indicate that the COPD condition may be undetected for the user 114 associated with the received audio input. In an embodiment, the modular neural network model may correspond to a generative cyclic autoencoder modular neural network (GCAE-MNN) model. The GCAE-MNN model may be trained based on a supervised learning algorithm. The encoding of the labeled data may be validated based on a regeneration of an input from the encoding.

In an embodiment, the GCAE-MNN model may include a statistical generative adversarial networks (GAN) model for the statistical analysis and augmentation of statistical extracted data The GAN model may include a generator model and discriminator model. The generator model may of the present disclosure may be configured to generate synthetic data that may be realistic based on received noise signal and a conditional vector. The generated synthetic data may be fed to the discriminator model. The discriminator mode may determine whether the generated synthetic data based is real or fake. In an embodiment, the GCAE-MNN model may further include a cyclic contractive autoencoder model. In an embodiment, the cyclic contractive autoencoder model may be configured to reduce a dimensionality associated with the set of audio features. In an embodiment, the cyclic contractive autoencoder model may be configured to fine-tune a set of hyper-parameters associated with the GCAE-MNN model.

In an embodiment, the AI model 112A, the RNN model 112B, and/or the modular neural network model 112C may be implemented using one or more neural network models. Each of the one or more neural network models may be a computational network or a system of artificial neurons, arranged in a plurality of layers, as nodes. The plurality of layers of the neural network model may include an input layer, one or more hidden layers, and an output layer. Each layer of the plurality of layers may include one or more nodes (or artificial neurons, represented by circles, for example). Outputs of all nodes in the input layer may be coupled to at least one node of hidden layer(s). Similarly, inputs of each hidden layer may be coupled to outputs of at least one node in other layers of the neural network model. Outputs of each hidden layer may be coupled to inputs of at least one node in other layers of the neural network model. Node(s) in the final layer may receive inputs from at least one hidden layer to output a result. The number of layers and the number of nodes in each layer may be determined from hyper-parameters of the neural network model. Such hyper-parameters may be set before, while training, or after training the neural network model on a training dataset.

Each node of the neural network model may correspond to a mathematical function (e.g., a sigmoid function or a rectified linear unit) with a set of parameters, tunable during training of the neural network model. The set of parameters may include, for example, a weight parameter, a regularization parameter, and the like. Each node may use the mathematical function to compute an output based on one or more inputs from nodes in other layer(s) (e.g., previous layer(s)) of the neural network model. All or some of the nodes of the neural network model may correspond to same or a different same mathematical function.

In training of the neural network model, one or more parameters of each node of the neural network model may be updated based on whether an output of the final layer for a given input matches a correct result based on a loss function for the neural network model. The above process may be repeated for same or a different input until a minima of loss function may be achieved, and a training error may be minimized. Several methods for training are known in art, for example, gradient descent, stochastic gradient descent, batch gradient descent, gradient boost, meta-heuristics, and the like.

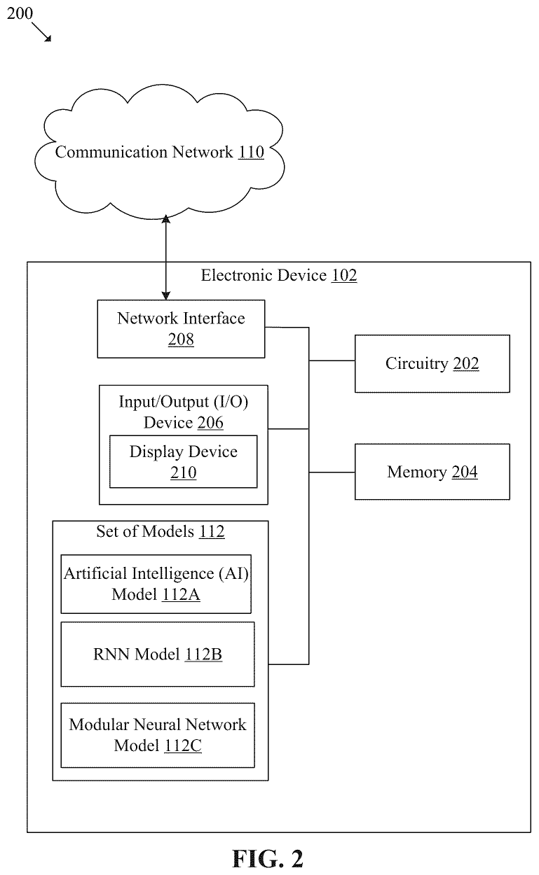

The neural network model may include electronic data, which may be implemented as, for example, a software component of an application executable on the electronic device 102 and/or the server 104. The neural network model may rely on libraries, external scripts, or other logic/instructions for execution by a computing device, such as, circuitry (e.g., circuitry 202 of FIG. 2) of the electronic device 102. The neural network model may include code and routines configured to enable the computing device, such as, the circuitry 202 of FIG. 2 to perform one or more operations for determination of the set of chronic obstructive pulmonary disease (COPD) metrics. Additionally, or alternatively, the neural network model may be implemented using hardware including a processor, a microprocessor (e.g., to perform or control performance of one or more operations), a field-programmable gate array (FPGA), or an application-specific integrated circuit (ASIC). Alternatively, in some embodiments, the neural network model may be implemented using a combination of hardware and software. Examples of the neural network model may include, but are not limited to, an encoder network model, a decoder network model, a transformer network model, a deep learning model, a convolution neural network model, a deep Bayesian neural network model, or a Generative Adversarial Network (GAN) model.

The network environment 100 may further include a distributed ledger (not shown in FIG. 1), that may be communicatively coupled to the electronic device 102 and/or the server 104, through the communication network 108. The distributed ledger may be a decentralized and distributed database system that may maintain an immutable record of data operations or transactions. The distributed ledger of the present disclosure may be configured to store encrypted audio samples. A node associated with distributed ledger may group a set of data operations as a block and may further link the block to a previous block of data operations to form a chain of a plurality of blocks. All blocks of data operations may be stored in a decentralized manner, whereby all participants or nodes may store all the plurality of blocks. Further, the distributed ledger may include an operating system which may allow for deployment of the group of smart contracts between multiple parties, for example, a user and a system.

The distributed ledger may be a chain of blocks which uses accounts as state objects and a state of each account can be tracked by the chain. Herein, the accounts represent identities of users, mining nodes, or automated agents. All the blocks of data operations or the smart contract may be associated with the accounts on the chain of blocks.

By way of example, and not limitation, the distributed ledger may be an Ethereum blockchain which may use accounts as state objects and a state of each account can be tracked by the Ethereum blockchain. Herein, the accounts may represent identities of users, mining nodes, or automated agents. All the blocks of data operations or the smart contract may be associated with the accounts on the Ethereum Blockchain. The scope of the disclosure may not be limited to the implementation of the distributed ledger as the Ethereum blockchain. The distributed ledger may be implemented as a Hyperledger blockchain or a Corda blockchain. Other implementations of the distributed ledger may be possible in the present disclosure, without a deviation from the scope of the present disclosure.

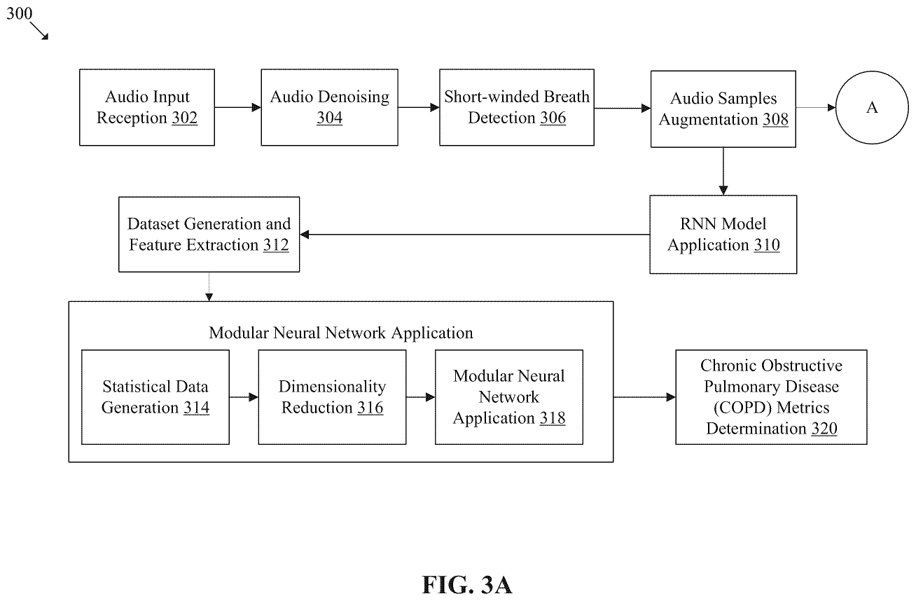

In operation, the electronic device 102 may be configured to receive the audio input associated with the user 114. For example, the electronic device 102 may include an application installed on the electronic device 102 to receive the audio input associated with the user 114. An instruction to determine whether the user 114 suffers from a COPD may also be received along with the audio input received by the electronic device 102 Details related to reception of the audio input are further provided, for example, in FIG. 3A.

The electronic device 102 may be configured to apply the Artificial Intelligence (AI) model 112A on the received audio input. Details related to the application of AI model 112A are further provided for example, in FIG. 3A. The electronic device 102 may be configured to detect the short-winded breath duration associated with the received audio input, based on the application of the AI model 112A on the received audio input. The short-winded breath duration may correspond to a time duration between an end of a first spoken word and a start of a second spoken word succeeding the first spoken word in the received audio input. Details related to the detection of the short-winded breath duration are further provided, for example, in FIG. 3A.

The electronic device 102 may be configured to detect the speaking pattern associated with the received audio input, based on the application of the AI model 112A on the received audio input and a geolocation of the user 114. Details related to the detection of the speaking pattern are further provided, for example, in FIG. 3A.

The electronic device 102 may be configured to apply the recurrent neural network (RNN) model 112B on audio samples associated with the received audio input, based on the detected short-winded breath duration and the detected speaking pattern. Details related to the application of the RNN model are further provided, for example, in FIG. 3A.

The electronic device 102 may be configured to reconstruct the set of short-winded breath audio samples based on the application of the RNN model 112B on the audio samples associated with the received audio input. Details related to the reconstruction of the set of short-winded breath audio samples are further provided, for example, in FIG. 3A.

The electronic device 102 may be configured to generate the audio sample dataset and the set of audio features associated with the generated audio sample dataset, based on the statistical analysis of the reconstructed set of short-winded breath audio samples. Details related to the generation of the audio sample dataset and the set of audio features are further provided, for example, in FIG. 3A.

The electronic device 102 may be configured to apply the modular neural network model 112C on the generated audio sample dataset and on the generated set of audio features. Details related to the application of the modular neural network model are further provided, for example, in FIG. 3A.

The electronic device 102 may be configured to determine the set of chronic obstructive pulmonary disease (COPD) metrics associated with the user 114, based on the application of the modular neural network model 112C on the generated audio sample dataset and the generated set of audio features. Details related to the determination of the COPD metrics are further provided, for example, in FIG. 3A.

The disclosed electronic device 102 may be used for chronic pulmonary disease (COPD) prediction from audio input based on the short-winded breath determination using artificial intelligence. The electronic device 102 may detect a COPD condition based on the short-winded breath duration. The prediction of the COPD based on the detected short-winded breath duration may be time-saving, cost effective, and non-invasive process for both the patient (such as, the user 114) and the doctor. Moreover, the electronic device 102 may detect different speaking patterns and may not get biased based on the different audio samples from different geographical regions. Furthermore, in some cases, the disclosed electronic device 102 may continuously monitor the user 114 and may record a set of audio inputs associated with the user 114 when a COPD condition is detected.

FIG. 2 is a block diagram that illustrates an exemplary electronic device of FIG. 1, in accordance with an embodiment of the disclosure. FIG. 2 is explained in conjunction with elements from FIG. 1. With reference to FIG. 2, there is shown the electronic device 102. The electronic device 102 may include circuitry 202, a memory 204, an input/output (I/O) device 206, a network interface 208, and the set of models 112. The input/output (I/O) device 206 may include a display device 210. The set of models may include the AI model 112A, RNN model 112B, and the modular neural network model 112C.

The circuitry 202 may include suitable logic, circuitry, and/or interfaces that may be configured to execute program instructions associated with different operations to be executed by the electronic device 102. The operations may include, for example, audio input reception, audio denoising, short-winded breath duration detection, audio samples augmentation, RNN model application, audio samples reconstruction, dataset generation and feature extraction, and COPD metrics determination. The circuitry 202 may include one or more processing units, which may be implemented as a separate processor. In an embodiment, the one or more processing units may be implemented as an integrated processor or a cluster of processors that perform the functions of the one or more specialized processing units, collectively. The circuitry 202 may be implemented based on a number of processor technologies known in the art. Examples of implementations of the circuitry 202 may be an X86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other control circuits.

The memory 204 may include suitable logic, circuitry, interfaces, and/or code that may be configured to store one or more instructions to be executed by the circuitry 202. The memory 204 may be configured to store the set of COPD metrics. Further, the memory 204 may be configured to store set of the audio input, the short-winded breath audio samples, the generated audio sample dataset, and the generated set of features. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, interfaces, and/or code that may be configured to receive an input and provide an output based on the received input. For example, the I/O device 206 may receive a first user input indicative of the audio input. The I/O device 206 may be further configured to display the determined COPD metric. The I/O device 206 may include the display device 210. Examples of the I/O device 206 may include, but are not limited to, a touch screen, a keyboard, a mouse, a joystick, a microphone, or a speaker.

The network interface 208 may include suitable logic, circuitry, interfaces, and/or code that may be configured to facilitate communication between the electronic device 102 and the server 104, via the communication network 108. The network interface 208 may also facilitate communication between the electronic device 102 and the distributed ledger (not shown in FIG. 1). The network interface 208 may be implemented by use of various known technologies to support wired or wireless communication of the electronic device 102 with the communication network. The network interface 208 may include, but is not limited to, an antenna, a radio frequency (RF) transceiver, one or more amplifiers, a tuner, one or more oscillators, a digital signal processor, a coder-decoder (CODEC) chipset, a subscriber identity module (SIM) card, or a local buffer circuitry.

The network interface 208 may be configured to communicate via wireless communication with networks, such as the Internet, an Intranet, a wireless network, a cellular telephone network, a wireless local area network (LAN), or a metropolitan area network (MAN). The wireless communication may be configured to use one or more of a plurality of communication standards, protocols and technologies, such as Global System for Mobile Communications (GSM), Enhanced Data GSM Environment (EDGE), wideband code division multiple access (W-CDMA), Long Term Evolution (LTE), 5th Generation (5G) New Radio (NR), code division multiple access (CDMA), time division multiple access (TDMA), Bluetooth, Wireless Fidelity (Wi-Fi) (such as IEEE 802.11a, IEEE 802.11b, IEEE 802.11g or IEEE 802.11n), voice over Internet Protocol (VoIP), light fidelity (Li-Fi), Worldwide Interoperability for Microwave Access (Wi-MAX), a protocol for email, instant messaging, and a Short Message Service (SMS).

The display device 210 may include suitable logic, circuitry, and interfaces that may be configured to display the set of COPD metrics. The display device 210 may be a touch screen which may enable a user (e.g., the user 114) to provide a user-input via the display device 210. The touch screen may be at least one of a resistive touch screen, a capacitive touch screen, or a thermal touch screen. The display device 210 may be realized through several known technologies such as, but not limited to, at least one of a Liquid Crystal Display (LCD) display, a Light Emitting Diode (LED) display, a plasma display, or an Organic LED (OLED) display technology, or other display devices. In accordance with an embodiment, the display device 210 may refer to a display screen of a head mounted device (HMD), a smart-glass device, a see-through display, a projection-based display, an electro-chromic display, or a transparent display. Various operations of the circuitry 202 for chronic pulmonary disease prediction from audio input based on short-winded breath determination using artificial intelligence are described further, for example, in FIGS. 3A and 3B.

FIGS. 3A and 3B are diagrams that collectively illustrate an exemplary processing pipeline for chronic pulmonary disease prediction from audio input based on short-winded breath determination using artificial intelligence, in accordance with an embodiment of the disclosure. FIGS. 3A and 3B are explained in conjunction with elements from FIG. 1 and FIG. 2. With reference to FIGS. 3A and 3B, there is shown an exemplary processing pipeline 300 that illustrates exemplary operations from 302 to 326. The exemplary operations 302 to 326 may be executed by any computing system, for example, by the electronic device 102 of FIG. 1 or by the circuitry 202 of FIG. 2.

At 302, an operation of audio input reception may be executed. The circuitry 202 may receive the audio input associated with the user 114. In an example, the electronic device 102 may include a microphone. The user 114 may be requested to speak in a direction of the microphone. The electronic device 102 may record live voice of the user 114 as the audio input. In another embodiment, the audio input associated with the user 114 may be a pre-recorded voice of the user 114 that may be stored in, for example, the database 106 and/or the memory 204. The circuitry 202 may retrieve the pre-recorded voice as the audio input.

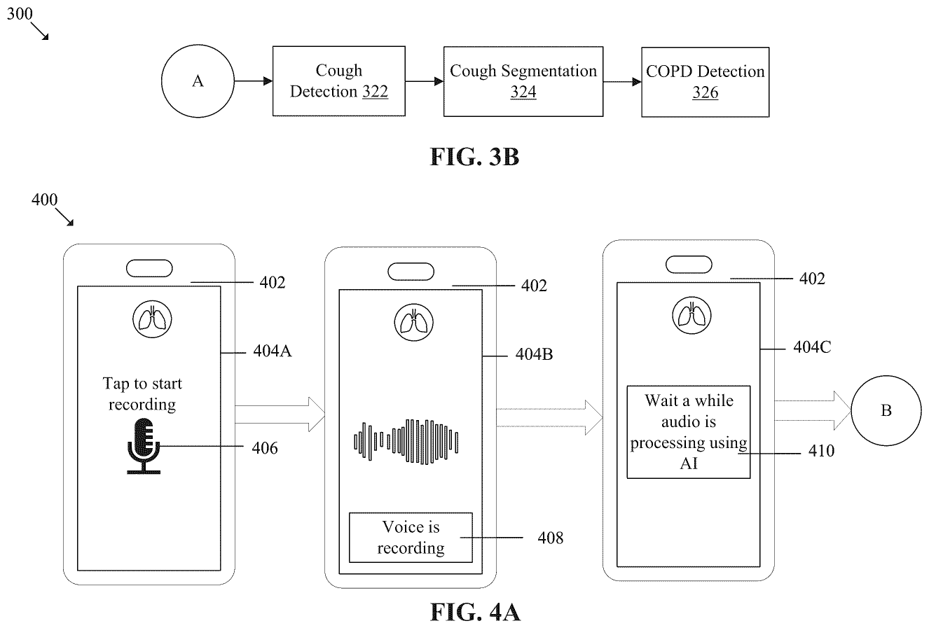

In an embodiment, the circuitry 202 may receive a video input associated with the user 114. The circuitry 202 may further extract the audio input from the received video input. Herein, the circuitry 202 may select a region of interest (ROI) from the received video input. For example, the ROI may correspond to a face of the user 114. Thereafter, the circuitry 202 may divide the received video input into a set of frames. The set of frames may include “N” number of frames. Further, the circuitry 202 may convert each frame of the set of frames into “N” number of patches. Finally, the circuitry 202 may further extract the audio input. Details related to the audio input reception are further provided, for example, in FIG. 4A.

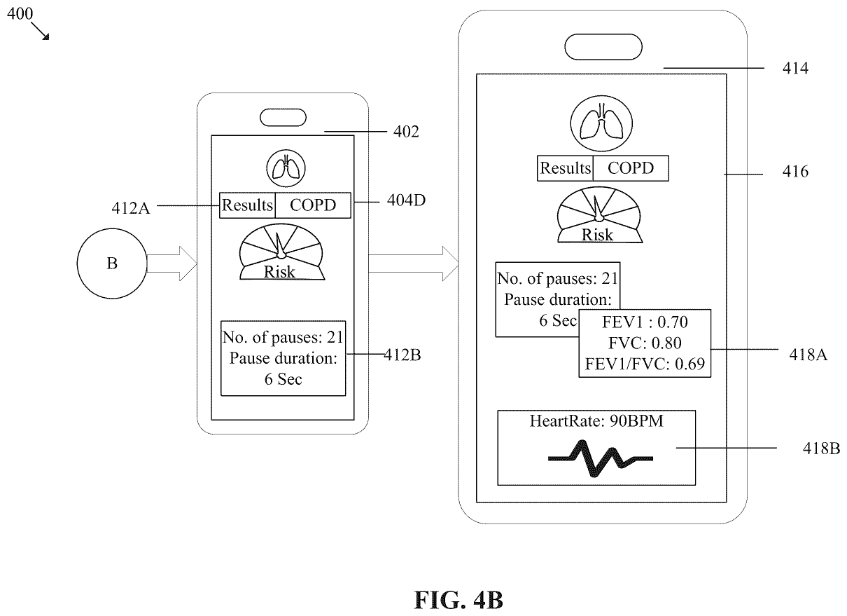

At 306, an operation for audio denoising may be executed. The circuitry 202 may be further configured to denoise the received audio input. It may be appreciated that the received audio input may be noisy. In an example, the received audio input may be an audio recording of the voice of the user 114. Herein, the received audio input may include the recorded voice of the user 114 and a background noise that may be prevalent in a background of the user 114, when the voice of the user 114 was recorded. Thus, the received audio input may be denoised. Details related to the audio denoising are provided, for example, in FIG. 5.

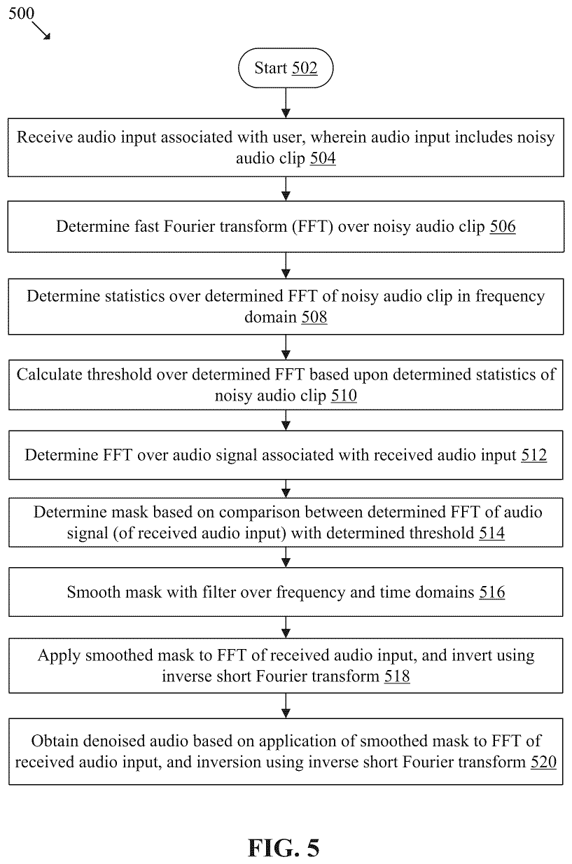

At 308, an operation for short-winded breath duration detection may be executed. The circuitry 202 may be configured to apply the AI model 112A on the denoised audio input. The circuitry 202 may be configured to detect the short-winded breath duration associated with the received audio input, based on the application of the AI model 112A on the denoised audio input. The short-winded breath duration may correspond to the time duration between the end of the first spoken word and the start of the second spoken word succeeding the first spoken word in the received audio input. It may be appreciated that a person such as, the user 114, may often take pauses while speaking. That is, the user 114 may take a pause after the end of the first spoken word and before the start of the second spoken word succeeding the first spoken word. The short-winded breath duration may be a duration of the pause taken by the user 114. The AI model 112A may be trained to detect the short-winded breath duration based on the received audio input. Details related to the detection of the short-winded breath duration are provided, for example, in FIG. 6.

In an embodiment, the circuitry 202 may be further configured to remove a set of non-COPD pauses from the received audio input. The detection of the short-winded breath duration may be further based on the removal of the set of non-COPD pauses. It may be appreciated that even a healthy person may take pauses while speaking. Such pauses may be of a shorter duration and may correspond to the non-COPD pauses. The non-COPD pauses may be pauses whose duration may be below a threshold. In case, the pause duration is below the threshold, the pause may correspond to the non-COPD pause and may be eliminated from the received audio input.

It may be noted that during deep breathing, an intensity of inspiratory breath sounds at a dominant frequency band (such as, “200” Hertz to “400” Hertz) may diminish over an upper and middle lung field in patients with COPD. Thus, the non-COPD pause may be the pauses in which an intensity of pause may be lesser than “200” Hertz. In an example, the non-COPD pauses may be removed from the received audio input, based on the intensity of the pause.

The circuitry 202 may be configured to detect a speaking pattern associated with the received audio input, based on the application of the AI model 112A on the received audio input and a geolocation of the user 114. It may be appreciated that the speaking pattern associated with the received audio input may vary geographically based on a language spoken in the geolocation of the user 114. Moreover, a breathless cycle or a duration of pauses taken by the user 114 may also vary based on the language spoken. For example, the detected speaking pattern may indicate how fast or how slow the user 114 talks, an accent of the user 114, a number of pauses taken by the user 114 while speaking, and the like. The AI model 112A may detect the speaking pattern associated with the received audio input based on the geolocation of the user 114. For example, the AI model 112A may be pre-trained to detect the speaking pattern based on predefined audio samples associated with people of different dialects, languages, and regions. Based on the application of the AI model 112A on the received audio input and on information related to the geolocation of the user 114, the speaking pattern associated with the received audio input may be determined. For example, a predefined audio sample associated with the geolocation of the user 114 may be determined by the AI model 112A. Further, the speaking pattern may be determined based on audio characteristics of the predefined audio sample.

At 308, an operation for audio samples augmentation may be executed. The circuitry 202 may be configured to augment the audio samples 110 associated with the received audio input. In an example, the audio samples 110 may be augmented based on an application of a speech augmentation technique. The augmentation of the audio samples 110 may enrich the audio samples 110 based on an addition of perturbations to the audio samples 110. The augmented audio samples may be then used to train the RNN model 112B.

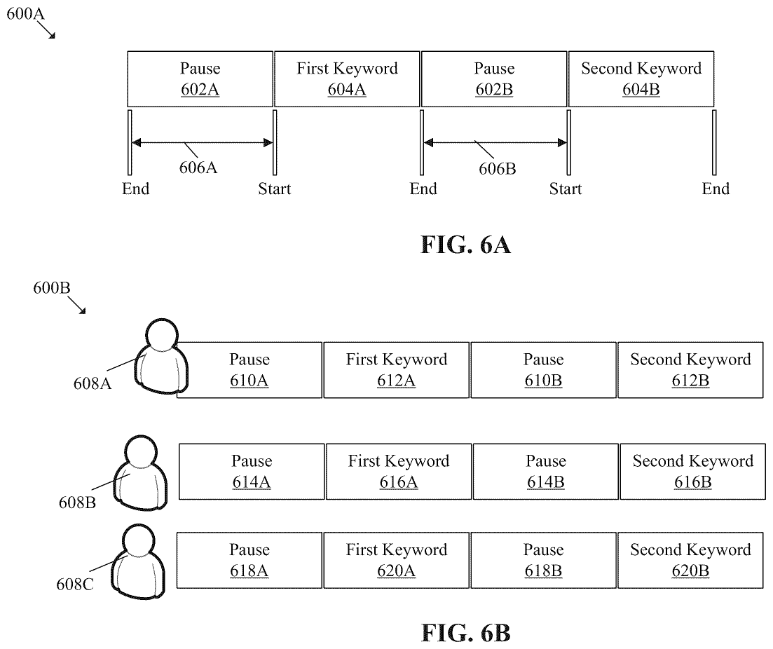

At 310, an operation for RNN model application may be executed. The circuitry 202 may be configured to apply the RNN model 112B on the augmented audio samples associated with the received audio input, based on the detected short-winded breath duration and the detected speaking pattern. The circuitry 202 may be configured to reconstruct the set of short-winded breath audio samples based on the application of the RNN model 112B on the audio samples 110 associated with the received audio input. As discussed, the received audio input may be denoised to remove noise components. Further, non-COPD pauses may be removed from the received audio input. In the process of denoising and removing non-COPD pauses, some samples of the audio samples associated with the received audio input may be lost. Moreover, some samples of the short-winded breath audio samples may not have sharp edges. In order to recover the audio samples that may been lost and to obtain sharp edges for the short-winded breath audio samples, the RNN model 112B may be applied on the audio samples 110. The RNN model 112B may reconstruct the set of short-winded breath audio samples. The RNN model 112B may reconstruct audio signals in case original audio signals are noisy or in case the original audio signals have skipped samples associated with breathlessness during recording of the audio data. Details related to the reconstruction of the set of short-winded breath audio samples are further provided, for example, in FIG. 7.

At 312, an operation for dataset generation and feature extraction may be executed. The circuitry 202 may be configured to generate the audio sample dataset and the set of audio features associated with the generated audio sample dataset, based on the statistical analysis of the reconstructed set of short-winded breath audio samples. The audio sample dataset may be generated based on the reconstructed set of short-winded breath audio samples. Each short-winded breath audio sample of the reconstructed set of short-winded breath audio samples may be analyzed statistically to extract the set of audio features. In an example, “parselmouth library” may be used to extract the set of audio features from the generated audio sample dataset.

In an embodiment, the set of audio features associated with the audio sample dataset may include at least one of, but not limited to, a mean audio frequency, a standard deviation of audio frequencies, a harmonics-to-noise ratio (HNR), a jitter, a shimmer, a format, a syllable per-group (SPG), a number of pauses per audio sample, a phonation time, a speech rate, an articulation rate, or an autism spectrum disorder (ASM) associated with the received audio input.

The mean audio frequency may be an average of audio frequency of the audio sample dataset. The standard deviation of audio frequencies may be calculated based on a distance of each audio frequency from the mean audio frequency. The HNR may represent a degree of acoustic periodicity and may be a ratio of periodic components to non-periodic components of the audio sample dataset. The jitter may be a modulation in periodicity of the received audio input and may be caused by irregular vocal fold vibration. Shimmer may also represent variations of frequency and amplitude in the received audio input and may be caused due instability in vocal fold vibration. The format may be a format of the audio sample dataset. The SPG may be an uninterrupted sound unit of a sonant or a vowel for each group of the reconstructed set of short-winded breath audio samples. The number of pauses per audio sample may be a total number of pauses taken by the user 114 in each audio sample. The phonation time may be time duration for which the user 114 may sustain phonation a sound associated with a vowel. The speech rate may be a speed at which the user 114 may speak and may be determined as a number of words spoken by the user 114 in a minute. The articulation rate may be also termed a speaking rate and may be defined as a pace at which a speech may be delivered by the user 114. The articulation rate may exclude the pauses taken by the user 114. The speech rate may also take into account a duration associated with a pause taken by the user 114. The autism spectrum disorder (ASM) associated with the received audio input may state whether the speaker such as the user 114 associated with the received audio input is autistic. It may be noted that the set of audio features associated with the audio sample dataset of a person having autism spectrum disorder may be different than the set of audio features associated with the audio sample dataset of a non-autistic person. For example, the person having autism spectrum disorder may take longer duration of pause or a greater number of pauses than the non-autistic person. Hence, the person having autism spectrum disorder may be falsely detected as having COPD. In order to mitigate the aforesaid issues, the set of audio features associated with the audio sample dataset may also indicate whether the person has ASM.

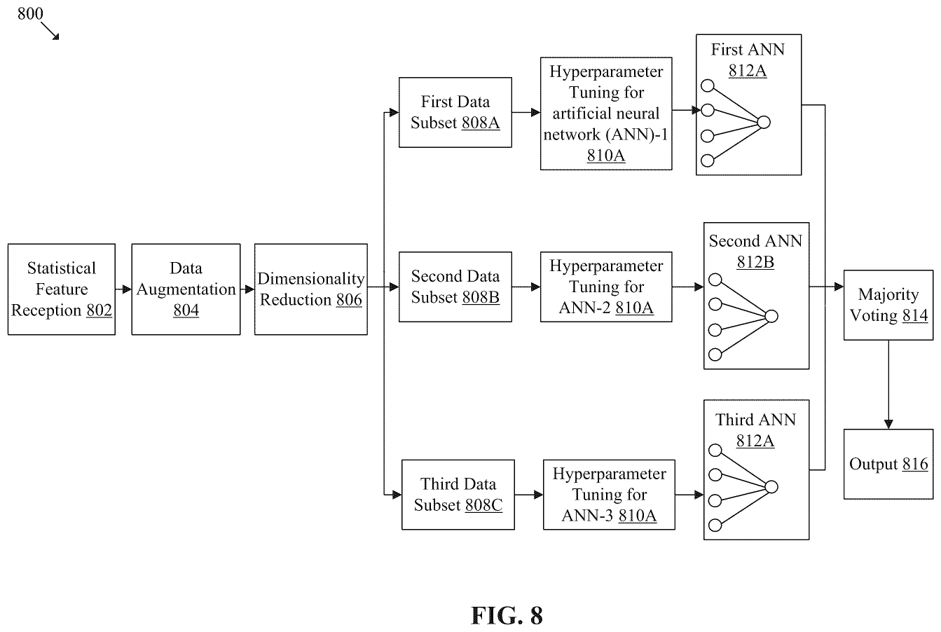

At 314, an operation for statistical data generation may be executed. The circuitry 202 may be configured to determine a statistical data based on a statistical analysis of the generated audio sample dataset. Herein, the statistical generative adversarial networks (GAN) model may be employed for the statistical analysis. Further, the GAN model may be used for statistical data augmentation and generation of statistical data from extracted statistical features. Details related to the statistical analysis and data augmentation are further provided, for example, in FIG. 9.

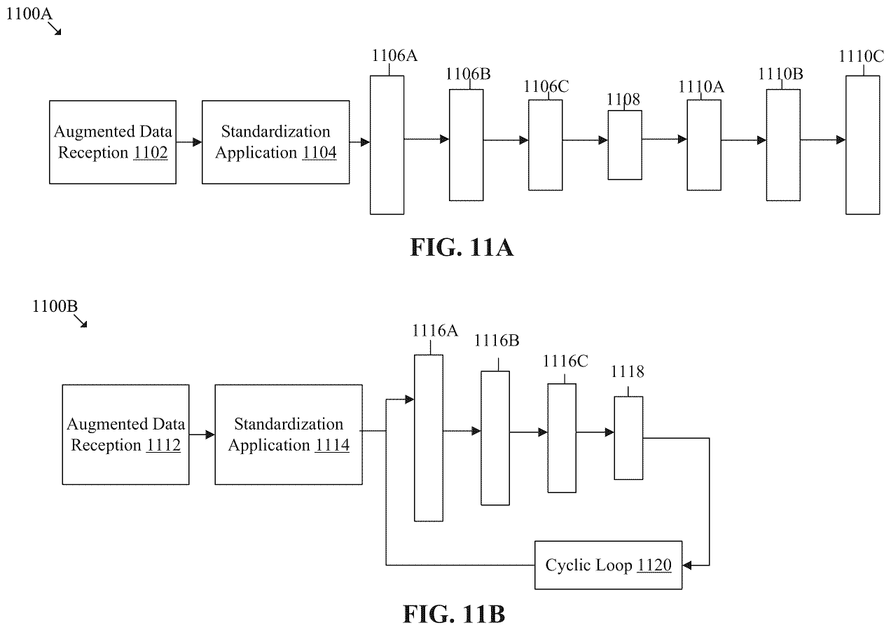

At 316, an operation for dimensionality reduction may be executed. The circuitry 202 may be configured to use a cyclic deep contractive autoencoder model to reduce a dimensionality associated with the set of audio features. It may be noted that the set of audio features may include a large number of audio features that may burden the modular neural network model 112C. Some of the audio features may be redundant and may be compressed to reduce the dimension of the set of audio features. Details related to the dimensionality reduction are further provided, for example, in FIG. 11.

At 318, an operation for modular neural network model application may be executed. The circuitry 202 may be configured to apply the modular neural network model 112C on the generated audio sample dataset and on the generated set of audio features. The modular neural network model 112C may be trained to determine the COPD metrics for the generated audio sample dataset and on the generated set of audio features. For example, the modular neural network model 112C may analyze the generated audio sample dataset and on the dimensionally reduced first set of audio features to determine the COPD metrics.

At 320, an operation for COPD metrics determination may be executed. The circuitry 202 may be configured to determine the set of COPD metrics associated with the user 114, based on the application of the modular neural network model 112C on the generated audio sample dataset and the generated set of audio features. The set of COPD metrics may be used to determine whether the user 114 associated with the audio input suffers from the COPD. The determined set of COPD metrics may be sent to an electronic device of a healthcare professional, such as, a doctor. The healthcare professional may analyze the determined set of COPD metrics to determine whether the user 114 associated with the audio input suffers from COPD.

In an embodiment, the set of COPD metrics associated with the user 114 may include at least one of, but not limited to, a COPD status, a COPD severity, COPD probability, a COPD infection level, COPD disease symptoms, a COPD sensitivity, a COPD level impacting other organs of the user, a probability of COPD level for developing other diseases, or COPD sensitivity level for other diseases. The COPD status may indicate whether the user 114 associated with the audio input suffers from COPD. In an example, the COPD status may be at least one of a yes status or a no status. The yes status may indicate that the user 114 suffers from COPD and no may indicate that the user 114 is free from the COPD. The COPD severity may indicate a severity level of COPD for the user 114. For example, the COPD severity may be at least one of mild, moderate, or severe. The COPD probability may indicate a probability that the user 114 associated with received audio input may suffer from COPD. The COPD infection level may provide an infection level for the user 114. For example, the COPD infection level may be at least one of, but not limited to, a first stage, a second stage, a third stage, and a fourth stage. The COPD disease symptoms may include shortness of breath, wheezing, chest tightness, a chronic cough, a respiratory infection, a lack of energy, weight loss, and the like. The COPD level impacting other organs of the user 114 may indicate a percentage by which organs, such as, lungs, kidney, and the like, may be affected due to the COPD. The probability of COPD level for developing other diseases may indicate a probability that the COPD may affect other organs such as, lungs, kidneys, and the like. The COPD sensitivity level for other diseases may indicate a level by which the user 114 may be affected by other diseases, such as, colds, flu, and pneumonia. The set of COPD metrics associated with the user 114 may be analyzed by the healthcare professional. The healthcare professional may prescribe medications based on the set of COPD metrics.

In an embodiment, the modular neural network model 112C may further configured to classify a health condition associated with the user as one of the COPD condition or the non-COPD condition. The COPD condition may determine that the user 114 associated with the received audio input may be suffering from the COPD and the non-COPD condition may state that the COPD condition is undetected for the user 114 associated with the received audio input.

In an embodiment, the circuitry 202 may be further configured to control recording of a set of audio inputs associated with the user 114, based on the determination of the set of COPD metrics associated with the user 114. Herein, the circuitry 202 may continuously monitor the set COPD metrics associated the user 114. In case, the set of COPD metrics associated the user 114 indicates that the COPD is detected, then the set of audio inputs associated with the user 114 may be recorded, otherwise the set of the set of audio inputs associated with the user 114 may not be recorded. The set of audio inputs may be audio signals associated with the user 114.

It may be noted that in some cases, cough may be used to determine whether the user 114 suffers from COPD. In such cases, the pipeline 300 may move from 308 to 322 (refer FIG. 3B). At 322, an operation for cough detection may be executed. The circuitry 202 may be configured to detect cough audio samples from the augmented audio samples associated with the received audio input. The person suffering COPD may cough when speaking. Hence, the augmented audio samples associated with the received audio input may include cough audio samples.

At 324, an operation for cough segmentation may be executed. The circuitry 202 may be configured to segment the detected cough audio samples. The segmentation may be performed to extract the detected cough audio samples. The segmentation may be performed based on an audio segmentation technique.

At 326, an operation for COPD detection may be executed. The circuitry 202 may be configured to classify a health condition associated with the user 114 as one of the non-COPD condition or the COPD condition, based on the segmentation of the detected cough audio samples. It may be noted that an intensity of cough, a duration of cough, and a cough sound classification associated with the segmented cough audio samples may be used to determine the health condition of the user 114. The health condition associated with the user 114 may be the non-COPD condition or a normal condition in case the user 114 is unaffected by COPD. The health condition associated with the user 114 may be the COPD condition in case the user 114 is affected by COPD.

Thus, the electronic device 102 may be used for chronic pulmonary disease (COPD) prediction from an audio input based on the short-winded breath determination using artificial intelligence. The electronic device 102 may detect a COPD condition based on the short-winded breath duration. Further, the prediction of the COPD based on the detected short-winded breath duration may be time-saving and self-alarming method for both the patient, such as, the user 114, and the doctor. Moreover, the electronic device 102 may detect and identify different speaking patterns and may not get biased based on the different audio samples from different geographical regions. Furthermore, in some cases, the disclosed electronic device 102 may continuously monitor the user 114 and may record a set of audio inputs associated with the user 114, when a COPD condition is detected.

FIGS. 4A and 4B are diagrams that together illustrate an exemplary scenario for chronic pulmonary disease prediction from audio input based on short-winded breath determination using artificial intelligence, in accordance with an embodiment of the disclosure. FIGS. 4A and 4B are described in conjunction with elements from FIG. 1, FIG. 2, FIG. 3A, and FIG. 3B. With reference to FIGS. 4A and 4B, there is shown an exemplary scenario 400. The exemplary scenario 400 may include a first electronic device 402, a first user interface (UI) 404A, a first UI element 406, a second UI 404B, a second UI element 408, a third UI 404C, a third UI element 410, a fourth UI 404D, a fourth UI element 412A, a fifth UI element 412B, a second electronic device 414, a fifth UI 416, a sixth UI element 418A, and a seventh UI element 418B. The first electronic device 404 may be associated with a patient, such as, the user 114. The second electronic device 414 may be associated with a healthcare profession, such as, a doctor. A set of operations associated the scenario 400 is described herein.

The first user interface 404A may be displayed on a display device, (such as, the display device 210 of FIG. 2) of the first electronic device 402. The first user interface 404A may provide the first UI element 406 that may be tapped (or selected) to start a recording of a speech of the user 114. Once, the first UI element 406 is tapped (or selected), the display device (such as, the display device 210 of FIG. 2) may display the second UI 404B. The second UI 404B may provide the second UI element 408. The second UI element 408 may notify the user 114 that a voice of the user 114 is being recorded. The recoding of the voice of the user 114 may be received as the audio input. Once, the audio input is received, the AI model 112A may be applied on the received audio input and the display device (such as, the display device 210 of FIG. 2) of the first electronic device 402 may display the third UI 404C. The third UI 404C may provide the third UI element 410. The third UI element 410 may notify the user 114 that the received audio input is being processed using artificial intelligence techniques to determine the set of COPD metrics. Thereafter, the display device (such as, the display device 210 of FIG. 2) of the first electronic device 402 may display the fourth UI 404D. The fourth UI 404D may include the fourth UI element 412A and the fifth UI element 412B. The fourth UI element 412A may notify the user 114 that the user 114 whether or not the user 114 suffers from “COPD”. For example, as shown in FIG. 4B, the fourth UI element 412A may display that the user 114 suffers from “COPD”. The fifth UI element 412B may provide the determined set of COPD metrics. For example, with reference to FIG. 4B, the fifth UI element 412B may indicate that a number of pauses for the user 114 is “21” and a pause duration is “6” seconds.

The determined set of COPD metrics may be transmitted to the second electronic device 414 associated with the healthcare profession. The display device (such as, the display device 210 of FIG. 2) of the second electronic device 414 may display the fifth UI 416. The fifth UI 416 may include the sixth UI element 418A and the seventh UI element 418B. The sixth UI element 418A may notify the healthcare processional of the determined set of COPD metrics for the user 114 and the sixth UI element 418A may notify the healthcare processional of a heartbeat of the user 114. The healthcare professional may view the determined set of COPD metrics and the heartbeat of the user 114 to decide a course of treatment.

Thus, the COPD may be detected for the user 114 in a non-invasive manner. Further, the healthcare processional may be notified of the determined set of COPD metrics. The healthcare processional may analyze the determined set of COPD metrics and may administer treatment remotely that may save time of the user 114 and the healthcare professional.

It should be noted that the scenario 400 of FIG. 4A and FIG. 4B is for exemplary purpose and should not be construed to limit the scope of the disclosure.

FIG. 5 is a flowchart that illustrates operations of an exemplary method for denoising audio input, in accordance with an embodiment of the disclosure. FIG. 5 is described in conjunction with elements from FIG. 1, FIG. 2, FIG. 3A, FIG. 3B, FIG. 4A, and FIG. 4B. With reference to FIG. 5, there is shown a flowchart 500. The flowchart 500 may include operations from 502 to 520 and may be implemented by the electronic device 102 of FIG. 1 or by the circuitry 202 of FIG. 2. The flowchart 500 may start at 502 and proceed to 504.

At 504, audio input associated with user 114 may be received, wherein the audio input may include a noisy audio clip. The circuitry 202 may be configured to receive the audio input associated with the user 114. The audio input may be a raw speech data associated with the user 114. In an example, the audio input includes a noisy audio clip associated with the user 114. Details related to the reception of the audio input are provided, for example, in FIG. 3A.

At 506, a fast Fourier transform (FFT) may be determined over the noisy audio clip. The circuitry 202 may be configured to determine the fast Fourier transform (FFT) over the noisy audio clip. In an embodiment, the noisy audio clip may be a noise component of the received audio input. In another embodiment, the noisy audio clip may be generated based on an additive white Gaussian noise (AWGN) signal. It may be appreciated that FFT may be an algorithm to calculate a discrete Fourier transform (DFT) over a signal (e.g., the noisy audio clip) to convert the signal to a frequency domain. The FFT of the received audio input may be determined to obtain spectral components associated with the received audio input.

At 508, statistics over the determined FFT of the noisy audio clip may be determined in the frequency domain. The circuitry 202 may be configured to determine statistics over the determined FFT of the noisy audio clip in the frequency domain. For example, statistics such as, a mean and a standard deviation of the noisy audio clip (e.g., the AWGN signal) may be calculated over the determined FFT of the noisy audio clip.

At 510, a threshold may be calculated over the determined FFT based upon the statistics of the noisy audio clip. The circuitry 202 may be configured to determine the threshold over the determined FFT based on the statistics of the noisy audio clip. It may be noted that a desired sensitivity of an algorithm for denoising the received audio input may be also considered for the determination of the threshold.

At 512, an FFT may be determined over an audio signal associated with the received audio input. The circuitry 202 may be configured to determine the fast Fourier transform (FFT) over the audio signal associated with the received audio input. The determination of the FFT over the audio signal may convert the audio signal to its spectral components in the frequency domain.

At 514, a mask may be determined based on a comparison between the determined FFT of the audio signal (of the received audio input) with the determined threshold. The circuitry 202 may be configured to determine the mask by comparing the determined FFT of the audio signal (associated with the received audio input) with the determined threshold. For example, the mean and standard deviation of the determined FFT of the audio signal may be calculated. Thereafter, the determined mean and standard deviation of the FFT of the audio signal (of the received audio input) may be compared with the determined mean and standard deviation of the FFT of the noisy audio clip to determine the mask.

At 516, the mask may be smoothed with a filter over frequency and time domains. The circuitry 202 may be configured to smooth the mask with a filter over the frequency and the time domains. For example, a smoothening filter may be applied on the mask over the frequency and the time domains to smoothen the mask.

At 518, the smoothed mask may be applied to the FFT of the received audio input. The circuitry 202 may be configured to apply the smoothened mask to the FFT of the received audio input. Thereafter, the circuitry 202 may be configured to invert the FFT of the received audio input using inverse short Fourier transform. It may be noted that the application of the smoothened mask to the FFT of the received audio input may remove noisy components from the FFT of the received audio input to obtain a denoised FFT. Further, the inversion of the FFT of the received audio input may convert the denoised FFT from the frequency domain to the time domain.

At 520, a denoised audio may be obtained based on the application of smoothed mask to the FFT of the received audio input, and further based on the inversion using the inverse short Fourier transform. The circuitry 202 may be configured to obtain the denoised audio based on the application of the smoothened mask to the FFT of the received audio input, and further based on the inversion of the FFT of the received audio input, using the inverse short Fourier transform. Control may pass to end.

Although the flowchart 500 is illustrated as discrete operations, such as, 504, 506, 508, 610, 512, 514, 516, 518, and 520, the disclosure is not so limited. Accordingly, in certain embodiments, such discrete operations may be further divided into additional operations, combined into fewer operations, or eliminated, depending on the implementation without detracting from the essence of the disclosed embodiments.

FIG. 6A is a diagram that illustrates an exemplary scenario for detection of short-winded breath duration, in accordance with an embodiment of the disclosure. FIG. 6A is described in conjunction with elements from FIG. 1, FIG. 2, FIG. 3A, FIG. 3B, FIG. 4A, FIG. 4B, and FIG. 5. With reference to FIG. 6A, there is shown an exemplary scenario 600A. The exemplary scenario 600A may include a pause 602A, a pause 602B, a first keyword 604A, a second keyword 604B, a short-winded breath duration 606A, and a short-winded breath duration 606B. A set of operations associated the scenario 600A is described herein.

For example, with reference to FIG. 6A, the AI model 112A may detect the pause 602A and the pause 602B. The short-winded breath duration 606A may be a duration of the pause 602A. The short-winded breath duration 606B may be a duration of the pause 602B. It may be noted that the short-winded breath duration 606B may correspond to the time duration between the end of the first keyword 604A and the start of the second keyword 604B.

It should be noted that the scenario 600A of FIG. 6A is for exemplary purpose and should not be construed to limit the scope of the disclosure.

FIG. 6B is a diagram that illustrates an exemplary scenario for detection of short-winded breath duration for a selected user, in accordance with an embodiment of the disclosure. FIG. 6B is described in conjunction with elements from FIG. 1, FIG. 2, FIG. 3A, FIG. 3B, FIG. 4A, FIG. 4B, FIG. 5, and FIG. 6A. With reference to FIG. 6B, there is shown an exemplary scenario 600B. The exemplary scenario 600B may include a user 608A, a user 608B, a user 608C, a pause 610A, a pause 610B, a first keyword 612A, a second keyword 612B, a pause 614A, a pause 614B, a first keyword 616A, a second keyword 616B, a pause 618A, a pause 618B, a first keyword 620A, a second keyword 620B. A set of operations associated the scenario 600B is described herein.

In an embodiment, the audio input may be associated with a set of users such as, a first user, a second user, and a third user. Only the first user may be interested in the detection of the set of COPD metrics. The second user and the third user may not be interested in the detection of the set of COPD metrics. The AI model 112A may exclude the audio input associated with the second user and the audio input associated with the third user during the detection of the short-winded breath duration.

For example, with reference to FIG. 6B, only the user 608A may be interested in COPD preprocessing. The user 608B and the user 608C may be uninterested in the COPD preprocessing. Therefore, the AI model 112A may detect the pause 610A and the pause 602B associated with the user 608A. A first short-winded breath duration may be a duration of the pause 610A. A second short-winded breath duration may be a duration of the pause 610B. It may be noted that the pause 614A, the pause 614B, the pause 618A, and the pause 618B may be undetected.

It should be noted that the scenario 600B of FIG. 6B is for exemplary purpose and should not be construed to limit the scope of the disclosure.

FIG. 6C is a diagram that illustrates an exemplary scenario for detection of short-winded breath duration for a plurality of users, in accordance with an embodiment of the disclosure. FIG. 6C is described in conjunction with elements from FIG. 1, FIG. 2, FIG. 3A, FIG. 3B, FIG. 4A, FIG. 4B, FIG. 5, FIG. 6A, and FIG. 6B. With reference to FIG. 6C, there is shown an exemplary scenario 600C. The exemplary scenario 600B may include a user 622A, a user 622B, a user 622C, a pause 624A, a pause 624B, a first keyword 626A, a second keyword 626B, a pause 628A, a pause 628B, a first keyword 630A, a second keyword 630B, a pause 632A, a pause 632B, a first keyword 634A, a second keyword 634B. A set of operations associated the scenario 600C is described herein.

In an embodiment, at least two users may be interested in the COPD processing and the AI model 112A may detect the short-winded breath duration for each of the at least two users. Further, the set of COPD metrics may be determined for each of the at least two users. For example, the first user and the second user may be interested in the detection of the set of COPD metrics. The third user may not be interested in the detection of the set of COPD metrics. In such a scenario, the AI model 112A may detect the short-winded breath duration for the first user and the second user. The set of COPD metrics may be determined for first user and the second user, separately. Thus, the present disclosure may allow processing of audio inputs associated with multiple patients for detection of the set of COPD metrics associated with each patient.

In an embodiment, short-winded data for multiple users may be saved and considered for training of the AI model 112A. Once a user opts for COPD processing, the trained AI model 112A may be applied to identify the corresponding user using statical parameters. In case a new user opts for the COPD processing, the AI model 112A may trigger a training of the AI model 112A for the new use. Herein, the new user may need to register for the COPD processing. It may be noted that short-winded statical parameters may be used continuous monitoring of the user during training of the AI model 112A. Thus, the user may be able to use the AI model 112A in any place irrespective of the environment in which the user is speaking.

For example, with reference to FIG. 6C, in an environment, the user 622A, the user 622B, and the user 622C may be speaking. However, only the user 622A and the user 622B may be interested in the COPD processing. The user 622C may be uninterested in the COPD processing. Therefore, the AI model 112A may detect the pause 624A and the pause 624B associated with the user 622A. A first short-winded breath duration associated with the user 622A may be a duration of the pause 624A. A second short-winded breath duration associated with the user 622A may be a duration of the pause 624B. Further, the AI model 112A may detect the pause 628A and the pause 628B associated with the user 622B. A first short-winded breath duration associated with the user 622B may be a duration of the pause 628A. A second short-winded breath duration associated with the user 622B may be a duration of the pause 628B. It may be noted that the pause 632A and the pause 632B may be undetected.

It should be noted that the scenario 600C of FIG. 6C is for exemplary purpose and should not be construed to limit the scope of the disclosure.

FIG. 7 is a diagram that illustrates an exemplary processing pipeline for reconstruction of a set of short-winded breath audio samples based on an application of Recurrent neural network (RNN) model, in accordance with an embodiment of the disclosure. FIG. 7 is explained in conjunction with elements from FIG. 1, FIG. 2, FIG. 3A, FIG. 3B, FIG. 4A, FIG. 4B, FIG. 5, FIG. 6A, FIG. 6B and FIG. 6C. With reference to FIG. 7, there is shown an exemplary processing pipeline 700 that illustrates exemplary operations from 702 to 714. The exemplary operations 702 to 714 may be executed by any computing system, for example, by the electronic device 102 of FIG. 1 or by the circuitry 202 of FIG. 2.

At 702, an operation for audio data reception may be executed. The circuitry 202 may be configured to receive audio data for the short-winded breath duration associated with the received audio input. The audio data may be the short-winded breath duration associated with the received audio input. That is, the audio data may be obtained based on an extraction of the short-winded breath duration from the received audio input. It may be noted that the audio data may be in a continuous time domain.

At 704, an operation for sampling of the audio data may be executed. The circuitry 202 may be configured to sample the received audio data. As discussed, the audio data may be in the continuous time domain. Hence, the audio data may be converted from the continuous time domain into a discreate time domain based on sampling of the audio data. Thus, the audio data may be converted into the set of short-winded breath audio samples.

At 706, an operation for RNN model application may be executed. The circuitry 202 may be configured to apply the Recurrent neural network (RNN) model 112B on the set of short-winded breath audio samples associated with the received audio input. The set of short-winded breath audio samples may be fed to the RNN model 112B for training purposes. Further, the RNN model 112B may analyze the set of short-winded breath audio samples to obtain a predicted signal. For example, a first subset of the short-winded breath audio samples (in discrete time domain) may be used as a training dataset for the RNN model 112B. Further, the trained RNN model 112B may be applied on a second subset of the short-winded breath audio samples to obtain the predicted signal.

At 708, an operation for obtaining a predicted signal may be executed. The circuitry 202 may be configured to obtain the predicted signal based on the application of the RNN model 112B on the set of short-winded breath audio samples. As discussed, some samples of the set of short-winded breath audio samples associated with the received audio input may be lost in the process of denoising and non-COPD pauses removal. Moreover, some samples of the set of short-winded breath audio samples may not have sharp edges. In order to recover the set of short-winded breath audio samples that may been lost and to obtain sharp edges for the short-winded breath audio samples, the RNN model 112B may be applied on the set of short-winded breath audio samples for reconstruction of such audio samples. The application of the RNN model 112 may generate the predicted signal in the discrete time domain.

At 710, an operation for conversion of the predicted signal may be executed. The circuitry 202 may be configured to convert the predicted signal from the discrete time domain into the continuous time domain.

At 712, an operation for generation of a final signal may be executed. The circuitry 202 may be configured to generate the final signal based on the conversion of the predicted signal from the discrete time domain into the continuous time domain. That is, the final signal may be the reconstructed set of short-winded breath audio converted into the continuous time domain.

At 714, an operation for dataset generation and feature extraction may be executed. The circuitry 202 may be configured to generate the audio sample dataset and the set of audio features associated with the generated audio sample dataset. Details related to the dataset generation and feature extraction are further provided for example, in FIG. 3A (at 312).

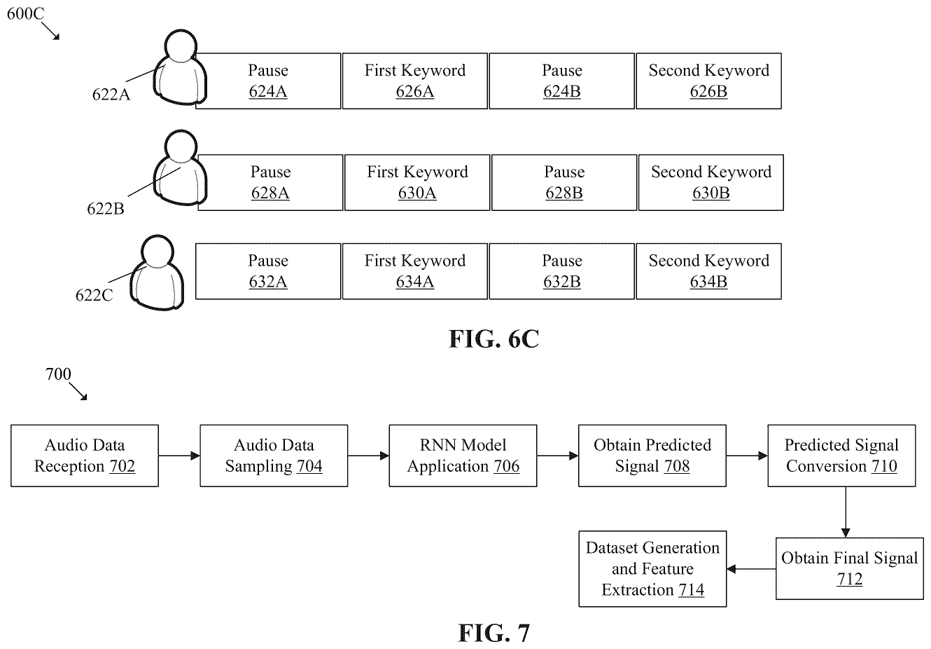

FIG. 8 is a diagram that illustrates an exemplary scenario for application of generative cyclic autoencoder modular neural network model, in accordance with an embodiment of the disclosure. FIG. 8 is described in conjunction with elements from FIG. 1, FIG. 2, FIG. 3A, FIG. 3B, FIG. 4A, FIG. 4B, FIG. 5, FIG. 6A, FIG. 6B, FIG. 6C, and FIG. 7. With reference to FIG. 8, there is shown an exemplary scenario 800. The exemplary scenario 800 may include a first data subset 808A, a second data subset 808B, a third data subset 808C, a first artificial neural network (ANN) 812A, a second ANN 812B, and a third ANN 812C. The exemplary scenario 800 may further include operations 802, 804, 806, 810A, 810B, 810C, 814, and 816. A set of operations associated the scenario 800 is described herein.

At 802, an operation for a set of audio feature reception may be executed. The circuitry 202 may be configured to receive the generated set of audio features. As discussed, the set of audio features associated with the generated audio sample dataset may be generated based on the statistical analysis of the reconstructed set of short-winded breath audio samples. Details related to the set of audio features generation are further provided, for example, in FIG. 3A (at 312).

At 804, an operation for data augmentation may be executed. The circuitry 202 may be configured to augment the set of audio features associated with the generated audio sample dataset. The augmentation of the set of audio features associated with the generated audio sample dataset may lead to an enrichment of the set of audio features. In an example, a dimensionality of the set of audio features may be “(23,25)” (that is, 23 rows and 25 columns). In one scenario, the “23” samples may be augmented by ten times to obtain “230” samples. In such case, the dimensionality of the augmented set of audio features may be “(300,25)”.

At 806, an operation for dimensionality reduction may be executed. The circuitry 202 may be configured to use the cyclic contractive autoencoder (CCAE) model to reduce the dimensionality associated with the augmented set of audio features. It may be noted that the augmented set of audio features may include a large number of audio features that may burden the modular neural network model 112C. Thus, the dimensionality of the augmented set of audio features may be reduced. The dimensionality reduction may correspond to a process for determination of a set of minimal features that may adequately represent all or most of the augmented set of audio features. The dimensionally reduced set of audio features may be converted into the first data subset 808A, the second data subset 808B, and the third data subset 808C.

In an embodiment, the dimensionally reduced set of audio features may be distributed equally in to the first data subset 808A, the second data subset 808B, and the third data subset 808C. In an example, the dimensionality of the set of audio features may be “(23, 25)”. The dimensionality of the augmented set of audio features may be “(300, 25)”. The dimensionality of the dimensionally reduced set of audio features may be “(300, 15)”. The dimensionally reduced set of audio features may be converted into the first data subset 808A, the second data subset 808B, and the third data subset 808C equally, such that the dimensionality of the each of the first data subset 808A, the second data subset 808B, and the third data subset 808C may be “(100, 15)”.

In another embodiment, the dimensionally reduced set of audio features may be distributed equally in to the first data subset 808A, the second data subset 808B, and the third data subset 808C based on a ranking of each sample of the dimensionally reduced set of audio features. Each sample of the dimensionally reduced set of audio features may be ranked. A number of samples (for example, “N” number of samples) may be selected based on the ranking and distributed equally into the first data subset 808A, the second data subset 808B, and the third data subset 808C in order include high quality samples. In some cases, a randomizer may be used to select top samples (for example, “N” number of samples) in order to reduce a load on the electronic device 102. In an example, the dimensionality of the set of audio features may be “(23, 25)”. The dimensionality of the augmented set of audio features may be “(300, 25)”. The dimensionality of the dimensionally reduced set of audio features may be “(300, 15)”. The dimensionally reduced set of audio features may be converted into the first data subset 808A, the second data subset 808B, and the third data subset 808C equally based on the ranking of each sample of the dimensionally reduced set of audio features, such that the dimensionality of each data subset may be “(80, 10)”.

In yet another embodiment, the dimensionally reduced set of audio features may be distributed equally in to the first data subset 808A, the second data subset 808B, and the third data subset 808C such that, the ranking of the samples of the first data subset 808A and the second data subset 808B may be higher than the ranking of the samples of the third data subset 808C. Herein, each sample of the dimensionally reduced set of audio features may be ranked. Thereafter, a first subset of the dimensionally reduced set of audio features in the order of highest ranking may be distributed into the first data subset 808A and the second data subset 808B. The samples of the dimensionally reduced set of audio features other than the first subset of the dimensionally reduced set of audio features may be selected as the third data subset 808C. In an example, the dimensionality of the set of audio features may be “(23,25)”. The dimensionality of the augmented set of audio features may be “(300,25)”. The dimensionality of the dimensionally reduced set of audio features may be “(300,15)”. The dimensionally reduced set of audio features may be converted into the first data subset 808A, the second data subset 808B, and the third data subset 808C equally based on ranking of each sample of the dimensionally reduced set of audio features, such that the dimensionality of each data subset may be “(100,15)”. Further, the ranking of the samples of the first data subset 808A and the second data subset 808B may be higher than the ranking of the samples of the third data subset 808C.

It may be noted that the cyclic contractive autoencoder (CCAE) model may be defined by a number of ways by the circuitry 202. In an example, a range of a number of layers may be defined to a select a number of layers for the CCAE model. A set of neurons may be defined to select the number of layers from the range of the number of layers. For example, 100, 50, or 20 neurons may be defined. Initially two layers, that is, an input layer and an output layer, may be defined. Further, a first set of neurons (e.g., M neurons) may be selected from the defined set of neurons for the input layer and a second set of neurons (e.g., N neurons) may be selected from the defined set of neurons for the output layer. For example, a set of “25” number of neurons may be selected from the defined set of neurons for the output layer. Thereafter, the CCAE model may be trained and a value of a loss function associated with the CCAE model may be determined based on the training. If the value of the loss function is determined as non-minimum (or is greater than a certain threshold value), then one or more layers from defined set of layers may be added to the CCAE model. Further, based on the value of the loss function not being minimum, a number of layers of the CCAE model may be increased and a number of neurons may be selected from the defined set of neurons may be selected for the additional layers of the CCAE model. For example, the number of neurons selected for the additional layers may lesser than or equal to the number of neurons in the input layer. The CCAE model may be retrained and a value of the loss function associated with the CCAE model may be determined. The training process of the CCAE model may be re-iterated and may continue until a value of the loss function becomes minimum (or becomes less than a certain threshold value).

At 810A, an operation for hyperparameter tuning of the first ANN 812A may be executed. At 810B, an operation for hyperparameter tuning of the second ANN 812B may be executed. At 810C, an operation for hyperparameter tuning of the third ANN 812C may be executed. The circuitry 202 may be configured to tune the hyperparameters of the first ANN 812A, the second ANN 812B, and the third ANN 812C. The first ANN 812A may be fed with the first data subset 808A to determine a first set of COPD metrics. The second ANN 812B may be fed with the second data subset 808B to determine a second set of COPD metrics. The third ANN 812C may be fed with the third data subset 808C to determine a third set of COPD metrics.

At 814, an operation for majority voting may be executed. The circuitry 202 may be configured to execute a majority voting operation on the outputs of the first ANN 812A, the second ANN 812B, and the third ANN 812C. For example, the first set of COPD metrics (i.e., the output of the first ANN 812A), the second set of COPD metrics (i.e., the output of the second ANN 812B), and the third set of COPD metrics (i.e., the output of the third ANN 812C) may be analyzed. Based on the analysis of the three sets of COPD metrics, and an application of a majority voting operation on the three sets of COPD metrics, the circuitry 202 may determine a final set of COPD metrics. In an example, the set of COPD metrics may be “1”, in case COPD is detected and “0” in case COPD is undetected, based on the received audio input. The first set of COPD metrics may be “1”, the second set of COPD metrics may be “0”, and the third set of COPD metrics may be “1”. Thus, the set of COPD metrics may be determined as “1” (that is COPD is detected) based on the majority voting. It may be noted that although the above example includes three sets of COPD metrics, the scope of the disclosure may not be so limited. A number of the sets of COPD metrics may be only two or more than two without departure from the scope of the disclosure.

At 816, an output may be generated. The circuitry 202 may be configured to generate the output (i.e., the final set of COPD metrics that may be determined at 814). Further, the circuitry 202 may display the generated output on the display device 210. For example, a text indicative of “COPD is detected” may be displayed on the display device 210 in case the final set of COPD metrics corresponds to a value of “1”.

It should be noted that the scenario 800 of FIG. 8 is for exemplary purpose and should not be construed to limit the scope of the disclosure.

FIG. 9 is a diagram that illustrate an exemplary processing pipeline for statistical data generation using statistical generative adversarial network model, in accordance with an embodiment of the disclosure. FIG. 9 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, and FIG. 8. With reference to FIG. 9, there is shown an exemplary processing pipeline 900 that illustrates exemplary operations from 902 to 912. The exemplary operations 902 to 912 may be executed by any computing system, for example, by the electronic device 102 of FIG. 1 or by the circuitry 202 of FIG. 2.

At 902, an operation for real data reception may be executed. The circuitry 202 may be configured to receive real data associated with the received audio input. For example, the real data may correspond to a raw audio signal associated with the received audio input.

At 904, an operation for mode specific normalization may be executed. The circuitry 202 may be configured to determine a normalized real data based on an execution of the mode specific normalization on the received real data. To normalize the real data, samples associated with the real data may be assigned to a set of modes via a gating network. Further, each sample may be estimated for normalization based on the mode to which the sample is assigned.

At 906, an operation for a noise signal reception may be executed. The circuitry 202 may be configured to receive the noise signal. For example, the received noise signal may correspond to an Additive White Gaussian Noise (AWGN) signal.

At 908, an operation for generator model application may be executed. The circuitry 202 may be configured to use a generator model to generate synthetic data and feed the generated synthetic data to a discriminator model. In an example, the generator model may include a first hidden layer, a second hidden layer, a fully connected layer, and a SoftMax activation function. The first hidden layer may include a first fully connected layer, a batch-normalization layer, and a leaky rectified linear unit (leaky ReLU) activation function. Similarly, the second hidden layer may include a second fully connected layer, a batch-normalization layer, and a leaky rectified linear unit (leaky ReLU) activation function. The generator model may learn a data distribution of the real data and a correlation between features from the real data to generate the synthetic data.

At 910, an operation for a discriminator model application may be executed. The circuitry 202 may be configured to apply the discriminator model on the normalized real data. In an example, the discriminator model may include a first hidden layer, a second hidden layer, and a fully connected layer. The first hidden layer may include a first fully connected layer, a leaky rectified linear unit (leaky ReLU) activation function, and a dropout layer. Similarly, the second hidden layer may include a second fully connected layer, a leaky rectified linear unit (leaky ReLU) activation function, and a dropout layer.

At 912, an operation to check for real or fake data may be executed. The circuitry 202 may be configured to use the discriminator model to determine whether the generated synthetic data received by the discriminator model is real or fake based on the application of the discriminator model on the normalized real data and on the generated synthetic data.

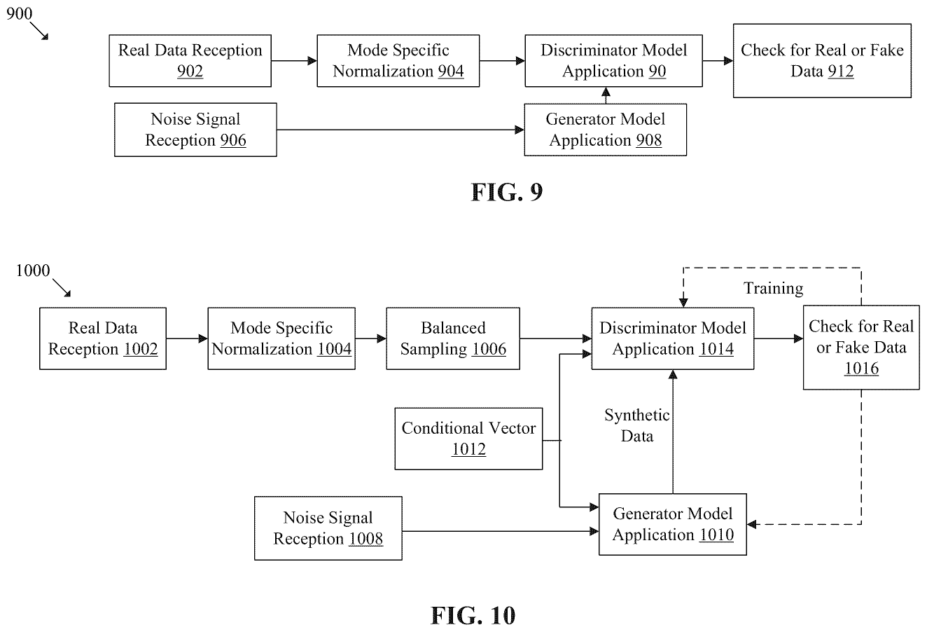

FIG. 10 is a diagram that illustrate an exemplary processing pipeline for statistical data generation using statistical generative adversarial network model, in accordance with an embodiment of the disclosure. FIG. 10 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, and FIG. 9. With reference to FIG. 10, there is shown an exemplary processing pipeline 900 that illustrates exemplary operations from 1002 to 1016. The exemplary operations 1002 to 1016 may be executed by any computing system, for example, by the electronic device 102 of FIG. 1 or by the circuitry 202 of FIG. 2.

At 1002, an operation for real data reception may be executed. The circuitry 202 may be configured to receive the real data associated with the audio input. For example, the real data may correspond to a raw audio signal associated with the received audio input.

At 1004, an operation for mode specific normalization may be executed. The circuitry 202 may be configured to determine a normalized real data based on an execution of the mode specific normalization on the received real data. Details related to the mode specific normalization are further provided, for example, in FIG. 9 (at 904).

At 1006, an operation for a balanced sampling may be executed. The circuitry 202 may be configured to apply a balanced sampling on the normalized real data to determine a balanced re. Based on the balanced sampling, a minority class associated with the normalized real data may be evenly sampled.

At 1008, an operation for a noise signal reception may be executed. The circuitry 202 may be configured to receive the noise signal. For example, the received noise signal may correspond to an Additive White Gaussian Noise (AWGN) signal.

At 1010, an operation for generator model application may be executed. The circuitry 202 may be configured to use the generator model to generate the synthetic data that may be realistic. The generation of the synthetic data by the generator model may be based on the received noise signal and a conditional vector 1012. The generated synthetic data may be fed to the discriminator model. Details related to the generator model are further provided for example, in FIG. 9.

At 1014, an operation for a discriminator model application may be executed. The circuitry 202 may be configured to apply the discriminator model on the balanced real data and the generated synthetic data, based on the conditional vector 1012. The discriminator model may determine whether the generated synthetic data based is real or fake.

At 1016, an operation to check for real or fake data may be executed. The circuitry 202 may be configured to use the discriminator model to determine whether the generated synthetic data is real or fake. For example, based on the application of the discriminator model on the balanced real data and the generated synthetic data, the circuitry 202 may determine whether the generated synthetic data is real or fake. The discriminator model and the generator model may be re-trained based on the determination whether the generated synthetic data is real or fake. For example, the generator model may be retained such that the generated synthetic data matches the real data. The generator model may learn a data distribution of the real data and a correlation between features of the real data and the generated synthetic data. The generator model may be considered as trained/fine-tuned when a probability that the discriminator model determines the generated synthetic data as fake is equal to or around 0.5. That is, when the discriminator model is unable to distinguish between the generated synthetic data and real data, the generator model may be considered as trained/fine-tuned.

FIG. 11A is a diagram that illustrates an exemplary scenario of cyclic deep contractive autoencoder model, in accordance with an embodiment of the disclosure. FIG. 11A is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, FIG. 9, and FIG. 10. With reference to FIG. 11A, there is shown an exemplary scenario 1100A. The exemplary scenario 1100A may include a first encoder layer 1106A, a second encoder layer 1106B, a third encoder layer 1106C, a bottleneck layer 1108, a first decoder layer 1110A, a second decoder layer 1110B, and a third decoder layer 1110C. The exemplary scenario 1100A may further include operations 1102 and 1104. A set of operations associated the scenario 1100A is described herein.

At 1102, an operation for augmented data reception may be executed. The circuitry 202 may be configured to receive the augmented data from the generative adversarial network (GAN) model. The augmented data may be the augmented set of audio features associated with the generated audio sample dataset. Details related to the generation of the augmented set of audio features are further provided, for example, in FIG. 8 (at 804).

At 1104, an operation for standardization application may be executed. The circuitry 202 may be configured to apply a standardization on the augmented data. Herein, a number of layers of the cyclic deep contractive autoencoder may be selected. A set of neurons may be defined for each layer of the selected number of layers (for example, the selected number of layers may be 100, 50, or 20). The first encoder layer 1106A may be defined as an input layer and the third decoder layer 1110C may be defined as an output layer. A first set of neurons may be selected from the defined set of neurons in the first encoder layer 1106A. For example, “M” number of neurons may be selected for the first encoder layer 1106A. A feature dimension of the first encoder layer 1106A may be “25” features. Further, “M′” number of neurons may be selected for the second encoder layer 1106B, such that “M′” may be lesser than or equal to “M”. A second set of neurons may be selected from the defined set of neurons for the third decoder layer 1110C. For example, “N” number of neurons may be selected for the output layer, that is, the third decoder layer 1110C. For the third encoder layer 1106C, “N′” number of neurons may be selected from the defined set of neurons such that, “N′” may less than “N”. The bottleneck layer 1108 may be used to reduce the feature dimension to obtain a reduced latent dimension output. In an example, a number of neurons lesser than “25” may be selected from the defined set of neurons in the output layer, that is, the third decoder layer 1110C. Thereafter, a loss associated with the cyclic contractive autoencoder (CCAE) model may be determined up to “25” iterations. Neurons may be selected for the layers such as, the first decoder layer 1110A and the second decoder layer 1110B, such that the loss may be minimum.

FIG. 11B is a diagram that illustrates an exemplary scenario of cyclic deep contractive autoencoder model with minimum losses, in accordance with an embodiment of the disclosure. FIG. 11B is described in conjunction with elements from FIG. 1, FIG. 2, FIG. 3A, FIG. 3B, FIG. 4A, FIG. 4B, FIG. 5, FIG. 6, FIG. 7, FIG. 8, FIG. 9, FIG. 10, and FIG. 11A. With reference to FIG. 11B, there is shown an exemplary scenario 1100B. The exemplary scenario 1100B may include a first encoder layer 1116A, a second encoder layer 1116B, a third encoder layer 1116C, a bottleneck layer 1118, and a cyclic loop 1120. The exemplary scenario 1100B may further include operations 1112 and 1114. A set of operations associated the scenario 1100B is described herein.

At 1112, an operation for augmented data reception may be executed. The circuitry 202 may be configured to receive the augmented data from the generative adversarial network (GAN). Details related to the augmented set of audio features are further provided, for example, in FIG. 8 (at 804).

At 1114, an operation for standardization application may be executed. The circuitry 202 may be configured to apply a standardization on the augmented data. Herein, a number of layers of the cyclic deep contractive autoencoder may be selected. Details related to the standardization application are further provided, for example, in FIG. 11A.

A set of neurons may be defined for each layer of the selected number of layers (for example, the selected number of layers may be 100, 50, or 20). The first encoder layer 1116A may be defined as an input layer. Details related to the selection of neurons for the first encoder layer 1116A, the second encoder layer 1116B, and the third encoder layer 1116C are further provided, for example, in FIG. 11A.

With reference to FIG. 11B, the bottleneck layer 1108 may be used to reduce the feature dimension to obtain the reduced latent dimension output. Thereafter, a value of a loss function associated with the cyclic contractive autoencoder (CCAE) model may be determined. The cyclic loop 1120 of feature compression may be run for compression of the feature dimension until a maximum compression may be achieved so that the value of the loss function may be minimum. After each loop, a compression ratio and a compression loss may be determined to control encoder layers such as, the first encoder layer 1116A, the second encoder layer 1116B, and the third encoder layer 1116C. Artificial neural network (ANN) based self-learning approaches may be used based on a type of samples. It may be noted that the number of layers may be dynamic based on a need of compression and/or dimension reduction. That is, the cyclic contractive autoencoder model may be trained and a value of the loss function associated with the cyclic contractive autoencoder model may be determined. If the value of the loss function is not minimum, then one or more layers from defined set of layers may be added such as, the second encoder layer 1116B, and the third encoder layer 1116C. The cyclic contractive autoencoder model may be retrained and the value of the loss function associated with the cyclic contractive autoencoder model may be determined. The cycle (of the cyclic loop 1120) may continue until the determined value of the loss function becomes minimum.

It should be noted that the scenarios 1100A and 1100B of FIGS. 11A and 11B, respectively, are for exemplary purposes and should not be construed to limit the scope of the disclosure.

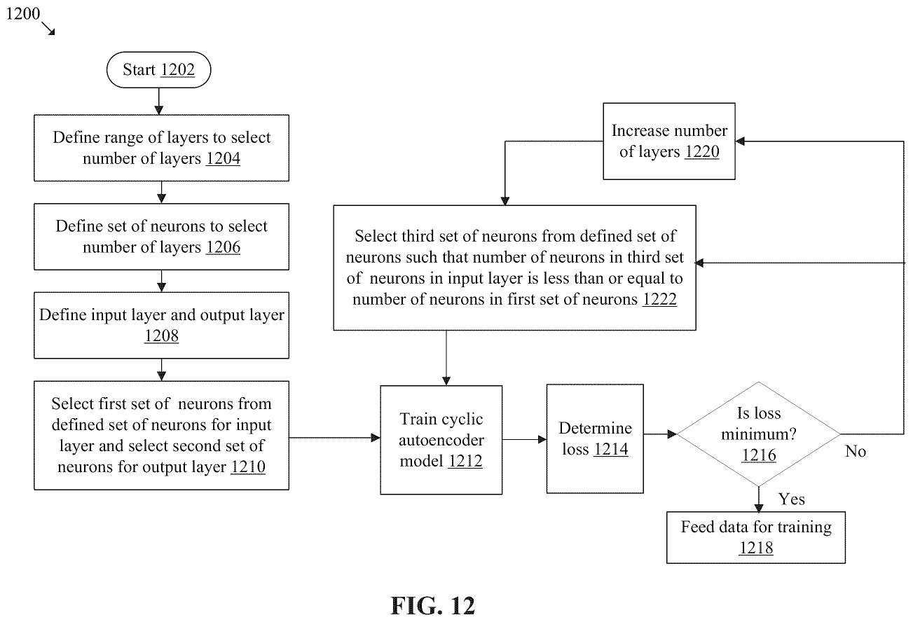

FIG. 12 is a flowchart that illustrates operations of an exemplary method for dimensionality reduction using cyclic contractive autoencoder model, in accordance with an embodiment of the disclosure. FIG. 12 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, FIG. 9, FIG. 10, FIG. 11A, and FIG. 11B. With reference to FIG. 12, there is shown a flowchart 1200. The flowchart 1200 may include operations from 1202 to 1222 and may be implemented by the electronic device 102 of FIG. 1 or by the circuitry 202 of FIG. 2. The flowchart 1200 may start at 1202 and proceed to 1204.

At 1204, a range of layers may be defined to select a number of layers. The circuitry 202 may be configured to define the range of layers to select the number of layers of the cyclic contractive autoencoder model. For example, the range of layers may be from “3” layers to “10” layers. That is, the cyclic contractive autoencoder model may have a minimum of “3” layers and a maximum of “10” layers.

At 1206, a set of neurons may be defined to select the number of layers. The circuitry 202 may be configured to define the set of neurons to select the number of layers of the cyclic contractive autoencoder model. For example, the defined set of neurons may include “100” neurons.

At 1208, an input layer and an output layer may be defined. The circuitry 202 may be configured to define the input layer and the output layer for the cyclic contractive autoencoder model. Details related to the input layer and the output layer are further provided, for example, FIG. 11A and FIG. 11B.

At 1210, a first set of neurons may be selected from the defined set of neurons for the input layer and a second set of neurons may be selected from the defined set of neurons for the output layer. The circuitry 202 may be configured to select a first set of neurons from the defined set of neurons for the input layer and select a second set of neurons from the defined set of neurons for the output layer. In an embodiment, “M” number of neurons may be selected from the defined set of neurons as the first set of neurons for the input layer and “N” number of neurons may be selected from the defined set of neurons as the second set of neurons for the second layer. In an example, “N” may be less than “25”. Details related to selection of the first set of neurons and the selection of the second set of neurons are provided, for example, in FIG. 11A and FIG. 11B.

At 1212, the cyclic contractive autoencoder model may be trained. The circuitry 202 may be configured to train the cyclic contractive autoencoder model. The artificial neural network (ANN) based self-learning approaches may be used based on the type of samples for training of the cyclic contractive autoencoder model.

At 1214, a loss (i.e., a value of the loss function) associated with the cyclic contractive autoencoder model may be determined. The circuitry 202 may be configured to determine the loss associated with the cyclic contractive autoencoder model. In an example, a loss function such as, a reconstruction loss function, a negative-log function, and the like, may be used to determine the loss associated with the cyclic contractive autoencoder model.

At 1216, a decision of whether the loss associated with the cyclic contractive autoencoder model is minimum may be determined. The circuitry 202 may be configured to determine whether the loss associated with the cyclic contractive autoencoder model is minimum.

In case the loss associated with the cyclic contractive autoencoder model is minimum, then control may pass to 1218. At 1218, the number of layers and the features associated with the number of layers may be used to feed training data. The circuitry 202 may be configured to feed the training data.

In case the loss associated with the cyclic contractive autoencoder model is not minimum, then control may pass to 1220. At 1220, the number of layers may be increased. The circuitry 202 may be configured to increase the number of layers. For example, a first intermediate layer may be added between the defined input layer and the defined output layer.

At 1222, a third set of neurons may be selected to increase the number of layers. The circuitry 202 may be configured to select a third set of neurons from the define set of neurons such that a number of neurons in the third set of neurons may be less than or equal to the number of neurons in the first set of neurons. For example, “M′” number of neurons may be selected as the third set of neurons for the intermediate layer, such that “M”′ may be lesser than or equal to “M”, where “M” is the number of neurons in the first set of neurons. Details related to the selection of the third set of neurons are further provided for example, in FIG. 11A and FIG. 11B. Thereafter, the cyclic contractive autoencoder model may be retrained and the steps 1212-1222 may be repeated till the defined set of neurons are processed and added to the CCAE model.

Although the flowchart 1200 is illustrated as discrete operations, such as, 1204, 1206, 1208, 1210, 1212, 1214, 1216, 1218, 1220, and 1222, the disclosure is not so limited. Accordingly, in certain embodiments, such discrete operations may be further divided into additional operations, combined into fewer operations, or eliminated, depending on the implementation without detracting from the essence of the disclosed embodiments.

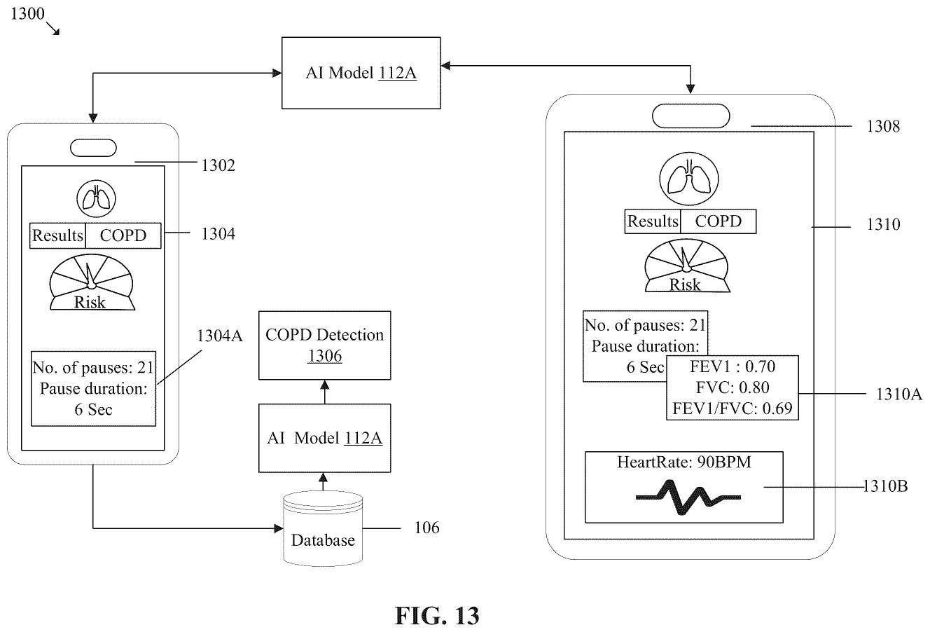

FIG. 13 is a diagram that illustrates an exemplary scenario for continuous monitoring of a user using artificial intelligence, in accordance with an embodiment of the disclosure. FIG. 13 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, FIG. 9, FIG. 10, FIG. 11A, FIG. 11B, and FIG. 12. With reference to FIG. 13 there is shown an exemplary scenario 1300. The exemplary scenario 400 may include a first electronic device 1302, a first UI 1304, a first UI element 1304A, the database 106, the AI model 112A, a second electronic device 1308, a second UI 1310, a second UI element 1310A, a third UI element 1310B, and a fourth UI element 1310C. The first electronic device 1302 may be associated with a patient, such as, the user 114. The second electronic device 1308 may be associated with a healthcare profession, such as, a doctor. The exemplary scenario 400 may further include an operation 1306 that may be executed by any computing system, for example, by the electronic device 102 of FIG. 1 or by the circuitry 202 of FIG. 2. A set of operations associated the scenario 1300 is described herein.

The first UI 1304 may be displayed on a display device, (such as, the display device 210 of FIG. 2) of the first electronic device 1302. The first UI 1304 includes the first UI element 1304A. The first UI element 1304A may provide the determined set of COPD metrics. For example, with reference to FIG. 4B, the first UI element 1304A may indicate that the number of pauses for the user 114 is “21” and the pause duration is “6” seconds.

The determined set of COPD metrics may be stored in the database 106. Further, the first electronic device 1302 may continuously monitor the user 114. Herein, audio data associated with the user 114 may be captured at regular intervals and stored in the database 106. For example, first audio data associated with the user 114 may be captured at a first time instant. Further, the captured first audio data may be stored in the database 106. The stored first audio data may be provided to the AI model 112A as an input. At 1306, an operation of COPD detection may be executed. Herein, the AI model 112A may be applied on the stored first audio data. Based on the application of the AI model 112A, the COPD metrics associated with the user 114 may be determined. The first electronic device 1302 may continuously monitor the user 114 and may thus, detect COPD in the user 114 beforehand based on breathlessness of the user 114.

Further, the determined COPD metrics may be transmitted to the second electronic device 1308 associated with the healthcare profession. The display device (such as, the display device 210 of FIG. 2) of the second electronic device 1308 may display the second UI 1310. The second UI 1310 may include the second UI element 1310A and the third UI element 1310B. The second UI element 1310A may notify the healthcare processional of the determined set of COPD metrics for the user 114, while the third UI element 1310B may notify the healthcare processional of the heartbeat of the user 114. The healthcare professional may view the determined set of COPD metrics and the heartbeat of the user 114 to decide a course of treatment.

It should be noted that the scenario 1300 of FIG. 13 is for exemplary purpose and should not be construed to limit the scope of the disclosure.

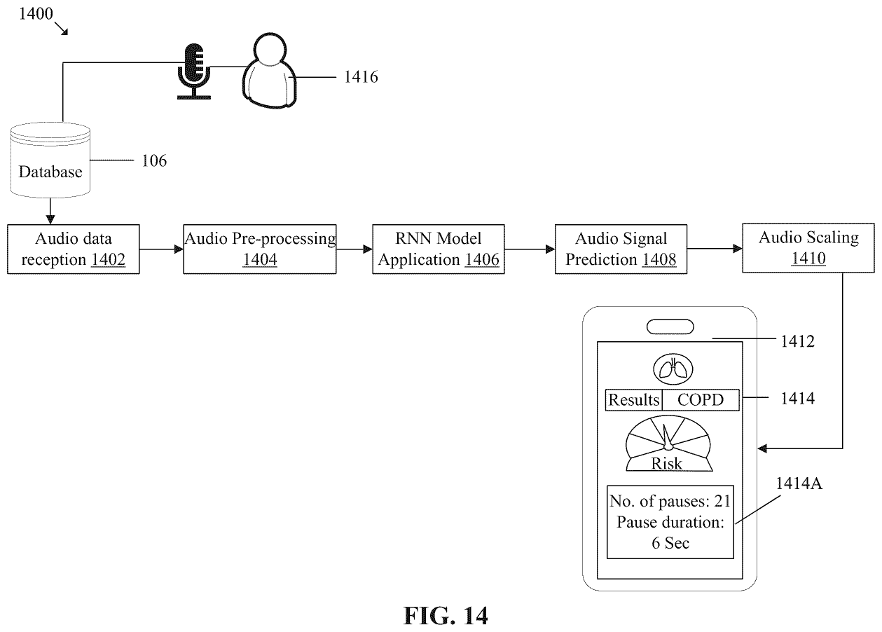

FIG. 14 is a diagram that illustrates an exemplary processing pipeline for continuous monitoring of a user using artificial intelligence, in accordance with an embodiment of the disclosure. FIG. 14 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, FIG. 9, FIG. 10, FIG. 11A, FIG. 11B, FIG. 12, and FIG. 13. With reference to FIG. 14 there is shown an exemplary processing pipeline 1400 that illustrates exemplary operations from 1402 to 1410. The exemplary operations 1402 to 1410 may be executed by any computing system, for example, by the electronic device 102 of FIG. 1 or by the circuitry 202 of FIG. 2. FIG. 14 further includes a first electronic device 1412, a first UI 1414, a first UI element 1414A, and a user 1416.

At 1402, an operation for audio data reception may be executed. Herein, the circuitry 202 may receive the audio data associated with the user 1416. In an embodiment, the audio data associated with the user 1416 may be recorded and stored in the database 106. Further, the circuitry 202 may request the database 106 for the audio data. The database 106 may verify the request and provide the audio data to the circuitry 202 based on the verification of the request. The provided audio data may be a continuous time signal.

At 1404, an operation for audio pre-processing may be executed. The circuitry 202 may pre-process the received audio data. Herein, the circuitry 202 may apply normalization and standardization on the received data

At 1406, an operation for RNN model application may be executed. The circuitry 202 may be configured to apply the RNN model 112B on the pre-processed audio data. Details related to the application of the RNN model 112B are further provided for example, in FIG. 3A.

At 1408, an operation for audio signal prediction may be executed. The circuitry 202 may be configured to predict audio signal based on the application of the RNN model 112B. Herein, the circuitry 202 may be configured to reconstruct the set of short-winded breath audio samples based on the application of the RNN model 112B.

At 1410, an operation for audio scaling may be executed. The circuitry 202 may be configured to scale the predicted audio signal into an original audio signal such as, the received audio data. The scaled audio signal may be used for COPD prediction. Based on the COPD prediction, the first UI 1414 may be displayed on a display device, (such as, the display device 210 of FIG. 2) of the first electronic device 1412. The first UI 1414 includes the first UI element 1414A. The first UI element 1404A may provide the determined set of COPD metrics.

It should be noted that the processing pipeline 1400 of FIG. 14 is for exemplary purpose and should not be construed to limit the scope of the disclosure

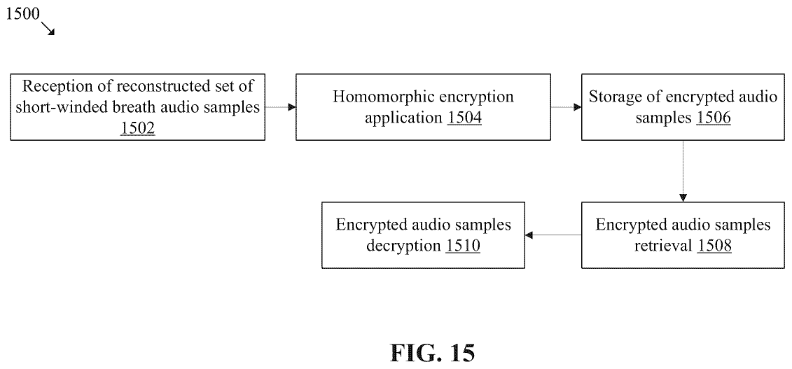

FIG. 15 is a diagram that illustrate an exemplary processing pipeline for encryption and decryption of reconstructed set of short-winded breath audio samples, in accordance with an embodiment of the disclosure. FIG. 15 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, FIG. 9, FIG. 10, FIG. 11A, FIG. 11B, FIG. 12, FIG. 13, and FIG. 14. With reference to FIG. 15, there is shown an exemplary processing pipeline 1500 that illustrates exemplary operations from 1502 to 1510. The exemplary operations 1502 to 1510 may be executed by any computing system, for example, by the electronic device 102 of FIG. 1 or by the circuitry 202 of FIG. 2.

At 1502, an operation for reception of reconstructed set of short-winded breath audio samples may be executed. In an embodiment, the circuitry 202 may be configured to receive the reconstructed set of short-winded breath audio samples from an electronic device of a healthcare provider. The healthcare provider may be a doctor, a hospital, and the like, that may have access to the reconstructed set of short-winded breath audio samples. Details related to the reconstructed set of short-winded breath audio samples, are provided, for example, in FIG. 3A.

At 1504, an operation of harmonics encryption application may be executed. In an embodiment, the circuitry 202 may be configured to apply the homomorphic encryption on the received reconstructed set of short-winded breath audio samples to determine encrypted audio samples. It may be noted that the harmonics encryption may be an encryption technique that may allow computations on encrypted data without decryption of the encrypted data. Thus, the computations may be executed on the encrypted data even without an access to a secret key associated with the encrypted data. However, the result of the computations may remain encrypted. The received reconstructed set of short-winded breath audio samples may be encrypted to determine the encrypted audio samples using homomorphic encryption so that the encrypted audio samples may be accessible to authorized personnel only.

At 1506, an operation for storage of the encrypted audio samples may be executed. In an embodiment, the circuitry 202 may be configured to store the encrypted audio samples on a distributed ledger (not shown). The distributed ledger may be a decentralized database including multiple distributed nodes that may store same data or a portion of common data. The transaction records associated with the encrypted audio samples may be included in a set of state objects, such as an initial state object and an updated version of the initial state object. Each state object may include a smart contract, a contract code (or rules of a transaction upon which parties to the transaction agree), and state properties (that may be updated when the transaction records may be updated based on transaction messages from the various parties). By way of example, and not limitation, the distributed ledger database may be a Corda blockchain, an Ethereum blockchain, or a Hyperledger blockchain.

At 1508, an operation for retrieval of the encrypted audio samples may be executed. In an embodiment, the circuitry 202 may be configured to retrieve, from the distributed ledger, the encrypted audio samples stored on the distributed ledger. In case, an entity or a person needs to access the encrypted audio samples, the encrypted audio samples may be retrieved from the distributed ledger. For example, an electronic device (such as, the second electronic device 414 associated with a doctor) may retrieve the encrypted audio samples stored on the distributed ledger.

At 1510, an operation for decryption of the encrypted audio samples may be executed. In an embodiment, the circuitry 202 may be configured to decrypt the encrypted audio samples using homomorphic decryption to determine decrypted audio samples. The set of COPD metrics may be determined further based on the decrypted audio samples. The encrypted audio samples may be decrypted via a private key associated with the encrypted audio samples. Thus, unauthorized users may be unable to decrypt the encrypted audio samples. The decrypted audio samples may be processed to determine the set of COPD metrics. Details related to the set of COPD metrics are provided, for example, in FIG. 3A.

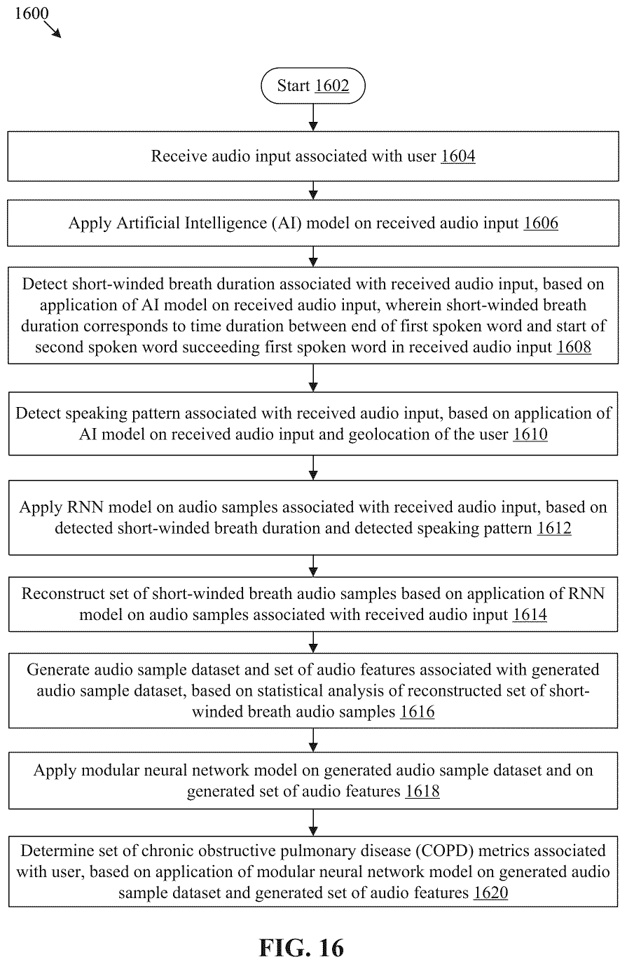

FIG. 16 is a flowchart that illustrates operations of an exemplary method for chronic pulmonary disease prediction from audio input based on short-winded breath determination using artificial intelligence, in accordance with an embodiment of the disclosure. FIG. 16 is described in conjunction with elements from FIG. 1, FIG. 2, FIG. 3A, FIG. 3B, FIG. 4A, FIG. 4B, FIG. 5, FIG. 6A, FIG. 6B, FIG. 6C, FIG. 7, FIG. 8, FIG. 9, FIG. 10, FIG. 11A, FIG. 11B, FIG. 12, FIG. 13, FIG. 14, and FIG. 15. With reference to FIG. 16, there is shown a flowchart 1600. The flowchart 1600 may include operations from 1602 to 1620 and may be implemented by the electronic device 102 of FIG. 1 or by the circuitry 202 of FIG. 2. The flowchart 1600 may start at 1602 and proceed to 1604.

At 1604, an audio input associated with a user may be received. The circuitry 202 may be configured to receive the audio input associated with the user 114. Details related to reception of the audio input are further provided for example, in FIG. 3A (at 302).

At 1606, the Artificial Intelligence (AI) model 112A may be applied on the received audio input. The circuitry 202 may be configured to apply the Artificial Intelligence (AI) model 112A on the received audio input. Details related to the application of AI model 112A are further provided for example, in FIG. 3A (at 306).

At 1608, a short-winded breath duration associated with the received audio input may be detected. The circuitry 202 may be configured to detect the short-winded breath duration associated with the received audio input, based on the application of the AI model 112A on the received audio input, wherein the short-winded breath duration may correspond to a time duration between an end of a first spoken word and a start of a second spoken word succeeding the first spoken word in the received audio input. Details related to short-winded breath duration are further provided for example, in FIG. 3A (at 306).

At 1610, a speaking pattern associated with the received audio input may be detected. The circuitry 202 may be configured to detect the speaking pattern associated with the received audio input, based on the application of the AI model 112A on the received audio input and a geolocation of the user 114. Details related to the speaking pattern are further provided for example, in FIG. 3A (at 306).

At 1612, the recurrent neural network (RNN) model 112B may be applied on audio samples associated with the received audio input. The circuitry 202 may be configured to apply the recurrent neural network (RNN) model 112B on audio samples associated with the received audio input, based on the detected short-winded breath duration and the detected speaking pattern. Details related to the application of the RNN model 112B are further provided for example, in FIG. 3A (at 310).

At 1614, a set of short-winded breath audio samples may be reconstructed. The circuitry 202 may be configured to reconstruct the set of short-winded breath audio samples based on the application of the RNN model 112B on the audio samples associated with the received audio input. Details related to reconstruction of the set of short-winded breath audio samples are further provided for example, in FIG. 3A (at 310).

At 1616, an audio sample dataset and a set of audio features associated with the generated audio sample dataset may be generated, based on a statistical analysis of the reconstructed set of short-winded breath audio samples. The circuitry 202 may be configured to generate the audio sample dataset and the set of audio features associated with the generated audio sample dataset, based on the statistical analysis of the reconstructed set of short-winded breath audio samples. Details related to generation of the audio sample dataset and the set of audio features are further provided for example, in FIG. 3A (at 312).

At 1618, the modular neural network model 112C may be applied on the generated audio sample dataset and on the generated set of audio features. The circuitry 202 may be configured to apply the modular neural network model 112C on the generated audio sample dataset and on the generated set of audio features. Details related to the application of the modular neural network model on the generated audio sample dataset and on the generated set of audio features are further provided for example, in FIG. 3A (at 314, 316, and 318).

At 1620, the set of chronic obstructive pulmonary disease (COPD) metrics associated with the user 114 may be determined, based on the application of the modular neural network model 112C on the generated audio sample dataset and the generated set of audio features. The circuitry 202 may be configured to determine the set of chronic obstructive pulmonary disease (COPD) metrics associated with the user 114, based on the application of the modular neural network model 112C on the generated audio sample dataset and the generated set of audio features. Details related to the COPD metrics are further provided for example, in FIG. 3A (at 320). Control may pass to end.

Although the flowchart 1600 is illustrated as discrete operations, such as, 1604, 1606, 1608, 1610, 1612, 1614, 1616, 1618, and 1620, the disclosure is not so limited. Accordingly, in certain embodiments, such discrete operations may be further divided into additional operations, combined into fewer operations, or eliminated, depending on the implementation without detracting from the essence of the disclosed embodiments.

Various embodiments of the disclosure may provide a non-transitory computer-readable medium and/or storage medium having stored thereon, computer-executable instructions executable by a machine and/or a computer to operate an electronic device (for example, the electronic device 102 of FIG. 1). Such instructions may cause the electronic device 102 to perform operations that may include receiving an audio input associated with a user (for example, the user 114 of FIG. 1). The operations may further include applying an Artificial Intelligence (AI) model (for example, the AI model 112A of FIG. 1) on the received audio input. The operations may further include detecting a short-winded breath duration associated with the received audio input, based on the application of the AI model 112A on the received audio input, wherein the short-winded breath duration may correspond to a time duration between an end of a first spoken word and a start of a second spoken word succeeding the first spoken word in the received audio input. The operations may further include detecting a speaking pattern associated with the received audio input, based on the application of the AI model 112A on the received audio input and a geolocation of the user 114. The operations may further include applying a recurrent neural network (RNN) model (for example, the RNN model 112B of FIG. 1) on audio samples associated with the received audio input, based on the detected short-winded breath duration and the detected speaking pattern. The operations may further include reconstructing a set of short-winded breath audio samples based on the application of the RNN model 112B on the audio samples associated with the received audio input. The operations may further include generating an audio sample dataset and a set of audio features associated with the generated audio sample dataset, based on a statistical analysis of the reconstructed set of short-winded breath audio samples. The operations may further include applying a modular neural network model (for example, the modular neural network model 112C of FIG. 1) on the generated audio sample dataset and on the generated set of audio features. The operations may further include determining a set of chronic obstructive pulmonary disease (COPD) metrics associated with the user 114, based on the application of the modular neural network model 112C on the generated audio sample dataset and the generated set of audio features.

Exemplary aspects of the disclosure may provide an electronic device (such as, the electronic device 102 of FIG. 1) that includes circuitry (such as, the circuitry 202). The circuitry 202 may be configured to receive an audio input associated with a user (for example, the user 114 of FIG. 1). The circuitry 202 may be configured to apply an Artificial Intelligence (AI) model (for example, the AI model 112A of FIG. 1) on the received audio input. The circuitry 202 may be configured to detect a short-winded breath duration associated with the received audio input, based on the application of the AI model 112A on the received audio input, wherein the short-winded breath duration may correspond to a time duration between an end of a first spoken word and a start of a second spoken word succeeding the first spoken word in the received audio input. The circuitry 202 may be configured to detect a speaking pattern associated with the received audio input, based on the application of the AI model 112A on the received audio input and a geolocation of the user 114. The circuitry 202 may be configured to apply a Recurrent neural network (RNN) model (for example, the RNN model 112B of FIG. 1) on audio samples associated with the received audio input, based on the detected short-winded breath duration and the detected speaking pattern. The circuitry 202 may be further configured to reconstruct a set of short-winded breath audio samples based on the application of the RNN model 112B on the audio samples associated with the received audio input. The circuitry 202 may be further configured to generate an audio sample dataset and a set of audio features associated with the generated audio sample dataset, based on a statistical analysis of the reconstructed set of short-winded breath audio samples. The circuitry 202 may be further configured to apply a modular neural network model (for example, the modular neural network model 112C of FIG. 1) on the generated audio sample dataset and on the generated set of audio features. The circuitry 202 may be further configured to determine a set of chronic obstructive pulmonary disease (COPD) metrics associated with the user 114, based on the application of the modular neural network model 112C on the generated audio sample dataset and the generated set of audio features.

In an embodiment, the circuitry 202 may be further configured to remove a set of non-COPD pauses from the received audio input, and wherein the detection of the short-winded breath duration may be further based on the removal of the set of non-COPD pauses.

In an embodiment, the AI model 112A may corresponds to a Self-Correcting Artificial Neural Network (SCANN) model.

In an embodiment, the circuitry 202 may be further configured to denoise the received audio input, and wherein the detection of the short-winded breath duration may be further based on the denoised audio input.

In an embodiment, the circuitry 202 may be further configured to augment the audio samples associated with the received audio input, and wherein the reconstruction of the set of short-winded breath audio samples may be further based on the augmentation of the audio samples associated with the received audio input.

In an embodiment, the circuitry 202 may be further configured to detect cough audio samples from the augmented audio samples associated with the received audio input. The circuitry 202 may be further configured to segment the detected cough audio samples. The circuitry 202 may be further configured to classify a health condition associated with the user as one of a normal condition or a COPD condition, based on the segmentation of the detected cough audio samples.

In an embodiment, the circuitry 202 may be further configured to control recording of a set of audio inputs associated with the user 114, based on the determination of the set of COPD metrics associated with the user 114.

In an embodiment, wherein the set of audio features associated with the audio sample dataset may be at least one of a mean audio frequency, a standard deviation of audio frequencies, a harmonics-to-noise ratio (HNR), a jitter, a shimmer, a format, a syllable per-group (SPG), a number of pauses per audio sample, a phonation time, a speech rate, an articulation rate, or an autism spectrum disorder (ASM) associated with the received audio input.

In an embodiment, the modular neural network model 112C may corresponds to a generative cyclic autoencoder modular neural network (GCAE-MNN) model.

In an embodiment, wherein the GCAE-MNN model may include a statistical generative adversarial networks (GAN) model for the statistical analysis.

In an embodiment, the GCAE-MNN model may further include a cyclic contractive autoencoder model.

In an embodiment, cyclic contractive autoencoder model may be configured to reduce a dimensionality associated with the set of audio features.

In an embodiment, the cyclic contractive autoencoder model may be configured to fine-tune a set of hyper-parameters associated with the GCAE-MNN model.

In an embodiment, modular neural network model 112C may be further configured to classify a health condition associated with the user 114 as one of a COPD condition or a non-COPD condition.

In an embodiment, the set of COPD metrics associated with the user 114 may include at least one of a COPD status, a COPD severity, COPD probability, a COPD infection level, COPD disease symptoms, a COPD sensitivity, a COPD level impacting other organs of the user, a probability of COPD level for developing other diseases, or COPD sensitivity level for other diseases.

In an embodiment, the circuitry 202 may be further configured to receive the reconstructed set of short-winded breath audio samples from a healthcare provider. The circuitry 202 may be further configured to apply homomorphic encryption on the received reconstructed set of short-winded breath audio samples to determine encrypted audio samples. The circuitry 202 may be further configured to store the encrypted audio samples on a distributed ledger. The circuitry 202 may be further configured to retrieve, from the distributed ledger, the encrypted audio samples stored on the distributed ledger. The circuitry 202 may be further configured to decrypt the encrypted audio samples using homomorphic decryption to determine decrypted audio samples, wherein the set of COPD metrics may be determined further based on the decrypted audio samples.

The present disclosure may also be positioned in a computer program product, which comprises all the features that enable the implementation of the methods described herein, and which when loaded in a computer system is able to carry out these methods. Computer program, in the present context, means any expression, in any language, code or notation, of a set of instructions intended to cause a system with information processing capability to perform a particular function either directly, or after either or both of the following: a) conversion to another language, code or notation; b) reproduction in a different material form.

While the present disclosure is described with reference to certain embodiments, it will be understood by those skilled in the art that various changes may be made, and equivalents may be substituted without departure from the scope of the present disclosure. In addition, many modifications may be made to adapt a particular situation or material to the teachings of the present disclosure without departure from its scope. Therefore, it is intended that the present disclosure is not limited to the embodiment disclosed, but that the present disclosure will include all embodiments that fall within the scope of the appended claims.

What is claimed is:

1. An electronic device, comprising:
circuitry configured to:
receive an audio input associated with a user;
apply an Artificial Intelligence (AI) model on the received audio input;
detect a short-winded breath duration associated with the received audio input, based on the application of the AI model on the received audio input, wherein
the short-winded breath duration corresponds to a time duration between an end of a first spoken word and a start of a second spoken word succeeding the first spoken word in the received audio input;
detect a speaking pattern associated with the received audio input, based on the application of the AI model on the received audio input and a geolocation of the user;
apply a recurrent neural network (RNN) model on audio samples associated with the received audio input, based on the detected short-winded breath duration and the detected speaking pattern;
reconstruct a set of short-winded breath audio samples based on the application of the RNN model on the audio samples associated with the received audio input;
generate an audio sample dataset and a set of audio features associated with the generated audio sample dataset, based on a statistical analysis of the reconstructed set of short-winded breath audio samples;
apply a modular neural network model on the generated audio sample dataset and on the generated set of audio features; and
determine a set of chronic obstructive pulmonary disease (COPD) metrics associated with the user, based on the application of the modular neural network model on the generated audio sample dataset and the generated set of audio features.

2. The electronic device according to claim 1, wherein the circuitry is further configured to remove a set of non-COPD pauses from the received audio input, and wherein
the detection of the short-winded breath duration is further based on the removal of the set of non-COPD pauses.

3. The electronic device according to claim 1, wherein the AI model corresponds to a Self-Correcting Artificial Neural Network (SCANN) model.

4. The electronic device according to claim 1, wherein the circuitry is further configured to denoise the received audio input, and wherein
the detection of the short-winded breath duration is further based on the denoised audio input.

5. The electronic device according to claim 1, wherein the circuitry is further configured to augment the audio samples associated with the received audio input, and wherein the reconstruction of the set of short-winded breath audio samples is further based on the augmentation of the audio samples associated with the received audio input.

6. The electronic device according to claim 5, wherein the circuitry is further configured to:
detect cough audio samples from the augmented audio samples associated with the received audio input;
segment the detected cough audio samples; and
classify a health condition associated with the user as one of a normal condition or a COPD condition, based on the segmentation of the detected cough audio samples.

7. The electronic device according to claim 1, wherein the circuitry is further configured to control recording of a set of audio inputs associated with the user, based on the determination of the set of COPD metrics associated with the user.

8. The electronic device according to claim 1, wherein the set of audio features associated with the audio sample dataset is at least one of a mean audio frequency, a standard deviation of audio frequencies, a harmonics-to-noise ratio (HNR), a jitter, a shimmer, a format, a syllable per-group (SPG), a number of pauses per audio sample, a phonation time, a speech rate, an articulation rate, or an autism spectrum disorder (ASM) associated with the received audio input.

9. The electronic device according to claim 1, wherein the modular neural network model corresponds to a generative cyclic autoencoder modular neural network (GCAE-MNN) model.

10. The electronic device according to claim 9, wherein the GCAE-MNN model includes a statistical generative adversarial networks (GAN) model for the statistical analysis.

11. The electronic device according to claim 9, wherein the GCAE-MNN model further includes a cyclic contractive autoencoder model.

12. The electronic device according to claim 11, wherein the cyclic contractive autoencoder model is configured to reduce a dimensionality associated with the set of audio features.

13. The electronic device according to claim 11, wherein the cyclic contractive autoencoder model is configured to fine-tune a set of hyper-parameters associated with the GCAE-MNN model.

14. The electronic device according to claim 1, wherein the modular neural network is further configured to classify a health condition associated with the user as one of a COPD condition or a non-COPD condition.

15. The electronic device according to claim 1, wherein the set of COPD metrics associated with the user includes at least one of a COPD status, a COPD severity, COPD probability, a COPD infection level, COPD disease symptoms, a COPD sensitivity, a COPD level impacting other organs of the user, a probability of COPD level for developing other diseases, or COPD sensitivity level for other diseases.

16. The electronic device according to claim 1, wherein the circuitry is further configured to:
receive the reconstructed set of short-winded breath audio samples from a healthcare provider;
apply homomorphic encryption on the received reconstructed set of short-winded breath audio samples to determine encrypted audio samples;
store the encrypted audio samples on a distributed ledger;
retrieve, from the distributed ledger, the encrypted audio samples stored on the distributed ledger; and
decrypt the encrypted audio samples using homomorphic decryption to determine decrypted audio samples, wherein
the set of COPD metrics is determined further based on the decrypted audio samples.

17. A method, comprising:
in an electronic device:
receiving an audio input associated with a user;
applying an Artificial Intelligence (AI) model on the received audio input;
detecting a short-winded breath duration associated with the received audio input, based on the application of the AI model on the received audio input, wherein
the short-winded breath duration corresponds to a time duration between an end of a first spoken word and a start of a second spoken word succeeding the first spoken word in the received audio input;
detecting a speaking pattern associated with the received audio input, based on the application of the AI model on the received audio input and a geolocation of the user;
applying a recurrent neural network (RNN) model on audio samples associated with the received audio input, based on the detected short-winded breath duration and the detected speaking pattern;
reconstructing a set of short-winded breath audio samples based on the application of the RNN model on the audio samples associated with the received audio input;
generating an audio sample dataset and a set of audio features associated with the generated audio sample dataset, based on a statistical analysis of the reconstructed set of short-winded breath audio samples;
applying a modular neural network model on the generated audio sample dataset and on the generated set of audio features; and
determining a set of chronic obstructive pulmonary disease (COPD) metrics associated with the user, based on the application of the modular neural network model on the generated audio sample dataset and the generated set of audio features.

18. The method according to claim 17, wherein the set of audio features associated with the audio sample dataset is at least one of a mean audio frequency, a standard deviation of audio frequencies, a harmonics-to-noise ratio (HNR), a jitter, a shimmer, a format, a syllable per-group (SPG), a number of pauses per audio sample, a phonation time, a speech rate, an articulation rate, or an autism spectrum disorder (ASM) associated with the received audio input.

19. The method according to claim 17, wherein the set of COPD metrics associated with the user includes at least one of a COPD status, a COPD severity, COPD probability, a COPD infection level, COPD disease symptoms, a COPD sensitivity, a COPD level impacting other organs of the user, a probability of COPD level for developing other diseases, or COPD sensitivity level for other diseases.

20. A non-transitory computer-readable medium having stored thereon, computer-executable instructions that when executed by an electronic device, causes the electronic device to execute operations, the operations comprising:
receiving an audio input associated with a user;
applying an Artificial Intelligence (AI) model on the received audio input;
detecting a short-winded breath duration associated with the received audio input, based on the application of the AI model on the received audio input, wherein
the short-winded breath duration corresponds to a time duration between an end of a first spoken word and a start of a second spoken word succeeding the first spoken word in the received audio input;
detecting a speaking pattern associated with the received audio input, based on the application of the AI model on the received audio input and a geolocation of the user;
applying a Recurrent neural network (RNN) model on audio samples associated with the received audio input, based on the detected short-winded breath duration and the detected speaking pattern;
reconstructing a set of short-winded breath audio samples based on the application of the RNN model on the audio samples associated with the received audio input;
generating an audio sample dataset and a set of audio features associated with the generated audio sample dataset, based on a statistical analysis of the reconstructed set of short-winded breath audio samples;
applying a modular neural network model on the generated audio sample dataset and on the generated set of audio features; and
determining a set of chronic obstructive pulmonary disease (COPD) metrics associated with the user, based on the application of the modular neural network model on the generated audio sample dataset and the generated set of audio features.
